(19) United States
(12) Patent Application Publication
(10) Pub. No.: US 2012/0269721 A1
(43) Pub. Date: Oct. 25, 2012

(54) TARGETED NANOCLUSTERS AND METHODS OF THEIR USE

(75) Inventors: Kevin C. Weng, San Jose, CA (US); Fanqing Frank Chen, Moraga, CA (US); Joe W. Gray, Lake Oswego, OR (US)

(73) Assignee: THE REGENTS OF THE UNIVERSITY OF CALIFORNIA, Oakland, CA (US)

(21) Appl. No.: 13/500,859

(22) PCT Filed: Oct. 8, 2010
(86) PCT No.: PCT/US10/52087

§ 371 Date: Jul. 2, 2012

Related U.S. Application Data
(60) Provisional application No. 61/250,793, filed on Oct. 12, 2009.

(51) Int. Cl. G01N 33/574; A61K 51/00; A61K 49/00; G01N 21/64; B82Y 15/00

(52) U.S. Cl. 424/1.11; 424/9.1; 435/7.23; 435/6.11; 977/773; 977/920

(57) ABSTRACT

This invention provides targeted nanoclusters comprising multiple polyvalent nanoparticle core units or nanoscaffolds, each nanoparticle core unit attached to multiple targeting moieties and multiple detectable moieties. The nanoclusters find use in a broad range of analytical assays, diagnostic assays and as targeted therapeutics.

### CROSS-REFERENCE TO RELATED APPLICATIONS

This application claims priority to U.S. Provisional Patent Application No. 61/250,793, filed on Oct. 12, 2009, hereby incorporated by reference in its entirety.

### STATEMENT OF GOVERNMENTAL SUPPORT

This invention was made with government support under Contract No. DE-AC02-05CH11231 awarded by the Department of Energy and LG013 Agilent Foundation Gift. The government has certain rights to this invention.

### FIELD OF THE INVENTION

The present invention relates to the field of compositions and methods of imaging and detection, using targeted nanoclusters or nanoaggregates comprising a plurality of nanoparticles attached to targeting moieties and detectable labels.

### BACKGROUND OF THE INVENTION

Imaging and detection techniques are widely used in molecular biology and clinical diagnosis. One particularly important application is characterization of cells or microscopic particles by flow cytometry or fluorescence-activated cell sorting (FACS). In flow cytometry, cells are stained with labeled antibody targeting specific antigen associated with the cells, and the label provides fluorescent signals when cells are suspended in a stream of fluid passing through an electronic detection apparatus. The signals, oftentimes in multiparametric format, can be interpreted as the levels of different antigens present in the cells and used to differentiate populations of cell of different characteristics. The capability of detecting low amount of certain marker in cells, and separating cell populations with high resolution, requires strong and quantitative binding of detection reagents. A conventional approach is to attach probes with strong signals to antibody without affecting the affinity and specificity of the antibody, which is technically challenging due to the linking chemistry and intrinsic steric limitation of the molecules.

Another important application is the characterization of biopsy samples collected from cancer patients in clinical pathology laboratories. Traditionally, tissues are fixed and preserved in paraffin, and examined in thin sections by light microscopy. Pathologists follow empirically established protocols that typically consist of the following steps: removal of paraffin, retrieval of antigens, application of primary antibody that targets the antigen of interest, application of enzyme-conjugated secondary antibody, application of chromogenic agents that reveal visible colors by enzymatic activation where antibodies localize, and lastly, counter-staining for feature enhancement. Such diagnostic procedures and laboratory operations in immunohistochemistry (IHC) have remained largely unchanged for more than a century. While antibody-antigen coupling occurs in the first binding reaction, it involves multiple time-consuming steps and each additional step embeds the risk of deviating from accuracy and consistency. More importantly, due to the biophysical properties of the reagents used, such as the most commonly adopted diaminobenzidine (DAB), quantitation and standardization of the assays often present significant challenges.¹ Assessments of various antigens and biomarkers in tumor tissues are important in determining tumor subtypes and response to therapy. As the body of knowledge in molecular signatures associated with tumor subtypes expanded vastly in recent years,²⁻⁴ the intrinsic disadvantages with the current method become serious obstacles for more efficient, quantitative, and consistent biomarker profiling. The emerging modalities in personalized medicine also call for a better way to correlate diagnostic profiling of individual tumors to prognosis and prediction of response to therapy. The area of ‘theranostics’ addresses these problems and aims to provide better strategies for connecting molecular diagnostics with targeted therapies that improve treatment outcomes for individual patients.

Sensitive immunodetection relies on multiple factors including specificity and affinity of the antibodies employed, and amplification of the signals from detected antigens. Several detection systems such as avidin-biotin complex (ABC), peroxidase anti-peroxidase (PAP), or polymer-based reagents have been used in traditional chromogenic techniques.⁵ They provide enhanced sensitivity through amplification; however, these systems also involve three or more steps, are not easy to quantify, and lack dynamic range.

Fluorescence-based immunodetection could potentially overcome the limitations and simplify the multi-step chromogenic methods by labeled primary or secondary antibodies; however, the trade-offs include need for optimizing conjugation for each primary antibody and loss of amplification due to non-crosslinked fluorophores on the secondary antibodies. Furthermore, the unstable and photobleachable nature of conventional fluorophores make them unpractical for long term storage and observation, especially in tissue banking for clinical studies. Semiconductor nanocrystals, e.g., quantum dots (QDs), that do not photobleach and offer broad spectral absorption and narrow emission profiles, enable excitation by a single low wavelength source and multiplex analysis.⁶⁻¹¹ The next generation of non-blinking QDs,^{12, 13} appear to be excellent candidates for immunodetection reagents. In fact, QDs have been demonstrated for use in immunohistochemistry by Nie et al.¹⁴ However, conjugation was reported to be inconsistent and required optimization for individual antibodies. The present invention provides a simple approach that offers avidity and detectable signal amplification benefits along with high sensitivity.

### SUMMARY OF THE INVENTION

The present invention provides a targeted nano-molecular complex, i.e., a nanocluster, comprised of stably associating a multiplicity of targeting moieties (e.g., antibodies and fragments thereof) and a multiplicity of detectable labels in an aggregation of a plurality of crosslinked nanoscaffold core structures. The targeted nanoclusters or nanoaggregates improve and simplify known methods for imaging and detection. The targeted nanoclusters or nanoaggregates described herein provide a higher sensitivity for detection due to an enhanced avidity effect by multiple anchoring points to a target and due to the amplification of detectable signal by multiple attachment to a plurality of detectable labels, within a single nanoparticle and multiplied by the aggregation of a plurality of crosslinked nanoparticle core units. Accordingly, enhanced signal amplification due to multiple reporting agents, e.g., for use in flow cytometry, immunocytochemistry/immunohistochemistry and in vivo imaging methods, are provided.

Accordingly, in one aspect, the invention provides compositions comprising a population of nanoclusters or nanoaggregates, the preponderance of nanoclusters or nanoaggregates in said population comprising a plurality of crosslinked nanoparticles, said nanoparticles comprising a nanoscaffold core structure having attached thereto:

a targeting moiety; and

a detectable label;

wherein the average number of nanoparticles in a nanocluster or nanoaggregate in said composition is about 2 or more.

In a related aspect, the invention provides compositions comprising a population of nanoclusters or nanoaggregates, the preponderance of nanoclusters or nanoaggregates in said population comprising a plurality of crosslinked nanoparticles, said nanoparticles comprising a nanoscaffold core structure having attached thereto:

a targeting moiety; and

a detectable label;

wherein the median number of nanoparticles in a nanocluster or nanoaggregate in said composition is about 2 or more.

In a further aspect, the invention provides a nanocluster or nanoaggregate comprising a plurality of crosslinked nanoparticles, said nanoparticles comprising a nanoscaffold core structure having attached thereto:

a targeting moiety; and

a detectable label.

With respect to the embodiments of the compositions, in some embodiments, the nanoclusters or nanoaggregates further comprise one or more crosslinkers covalently linking the multiple nanoscaffold core structures. In various embodiments, the median number or average number of nanoparticles in a nanocluster or nanoaggregate is about 2, about 3 or more, about 4 or more, about 5 or more, about 6 or more, about 7 or more, about 8 or more, about 9 or more, or about 10 or more. In some embodiments, the number of nanoparticles in a nanocluster or nanoaggregate is about 2, about 3 or more, about 4 or more, about 5 or more, about 6 or more, about 7 or more, about 8 or more, about 9 or more, or about 10 or more. In some embodiments, the nanoscaffold core structures have an average diameter that is less than about 100 nm, for example, an average diameter that is less than about 90 nm, 80 nm, 70 nm, 60 nm, 50 nm, 40 nm, 30 nm, 20 nm, or smaller. In some embodiments, the nanoscaffold core structures bear on average at least 3, or at least 4, or at least 5, or at least 10, or at least 20, or at least 50, or at least 100 or at least 500, or at least 1000 targeting moieties. In various embodiments, the targeting moieties can be the same or different; the targeting moieties can specifically or preferentially bind to the same or different target antigens or biomarkers. In some embodiments, the targeting moieties are all the same. In some embodiments, the targeting moieties attached to a nanoscaffold comprise a plurality of different targeting moieties. In some embodiments, the targeting moieties attached to a nanoscaffold comprise at least two different targeting moieties that bind different targets/epitopes on a target cell.

In some embodiments, the nanoscaffold core structures bear on average at least 3, or at least 4, or at least 5, or at least 10, or at least 20, or at least 50, or at least 100 or at least 500, or at least 1000 detectable labels. In various embodiments, the detectable labels can be the same or different. In some embodiments, the detectable labels are all the same. In some embodiments, the detectable labels comprise a plurality of different detectable labels. In some embodiments, the detectable labels attached to a nanoscaffold comprise at least two different detectable labels, each label detectable by a different detection modality. In some embodiments, the nanoscaffold core structure is selected from the group consisting of a lipidic particle, a dendrimer, a hyperbranched polymer, a metal particle, a particle comprising a group II, III, or IV material, a polymeric nanoparticle, a glass nanoparticle, a quartz nanoparticle, a viral nanoparticle, a silicon oxide nanoparticle and a silica nanoparticle. In some embodiments, the nanoscaffold core structure comprises a lipidic particle selected from the group consisting of a liposome, a micelle, a lipid vesicle and a multilamellar vesicle. In some embodiments, the nanoscaffold core structure is a lipid vesicle.

In some embodiments, the targeting moiety specifically or preferentially binds to a cancer or tumor marker. In some embodiments, the targeting moiety selectively or preferentially binds to a cancer marker selected from Her2/neu, 5-alpha reductase, α-fetoprotein, AM-1, APC, APRIL, BAGE, β-catenin, Bc12, bcr-abl (b3a2), CA 125, CASP-8/FLICE, Cathepsins, CD19, CD20, CD21, CD23, CD22, CD38, CD33, CD35, CD44, CD45, CD46, CD5, CD52, CD55, CD59 (791Tgp72), CDC27, CDK4, CEA, c-myc, COX-2, Cytokeratin, DCC, DcR3, E6/E7, EGFR, EMBP, Ena78, Estrogen Receptor (ER), FGF8b and FGF8a, FLK 1/KDR, Folic Acid Receptor, G250, GAGE-Family, gastrin 17, Gastrin-releasing hormone (bombesin), GD2/GD3/GM2, GnRH, GnTV, gp100/Pme117, gp-100-in4, gp15, gp75/TRP-1, hCG, Heparanase, Her3, HMTV, Hsp70, hTERT (telomerase), IGFR1, IL 13R, iNOS, Ki 67, KIAA0205, K-ras, H-ras, N-ras, KSA (CO17-1A), LDLR-FUT, MAGE Family (MAGE1, MAGE3, etc.), Mammaglobin, MAP17, Melan-A/MART-1, mesothelin, MIC A/B, MT-MMP's, such as MMP2, MMP3, MMP7, MMP9, Mox1, Mucin, such as MUC-1, MUC-2, MUC-3, and MUC-4, MUM-1, NY-ESO-1, Osteonectin, p15, P170/MDR1, p53, p97/melanotransferrin, PAI-1, PDGF, Plasminogen (uPA), PRAME, Probasin, Progenipoietin, Progesterone Receptor (PR), PSA, PSM, RAGE-1, Rb, RCAS1, SART-1, SSX gene family, STAT3, STn (mucin assoc.), TAG-72, TGF-α, TGF-β, Thymosin β-15, IFN-γ, TPA, TPI, TRP-2, Tyrosinase, VEGF, ZAG, p16INK4, Glutathione and S-transferase. In some embodiments, the targeting moiety selectively or preferentially binds to Her2/neu.

In some embodiments, the targeting moiety specifically or preferentially binds to a cell from a cancer selected from the group consisting of breast cancer, colorectal cancer, NSCLC, lung cancer, bone cancer, pancreatic cancer, skin cancer, cancer of the head or neck, cutaneous melanoma, intraocular melanoma, uterine cancer, ovarian cancer, rectal cancer, anal region cancer, stomach cancer, gastric cancer, colon cancer, breast cancer, uterine cancer, fallopian tube carcinoma, endometrial carcinoma, cervical carcinoma, vaginal carcinoma, vulval carcinoma, Hodgkin's Disease, esophagus cancer, small intestine cancer, endocrine system cancer, thyroid gland cancer, parathyroid gland cancer, adrenal gland cancer, soft tissue sarcoma, urethral cancer, penis cancer, prostate cancer, bladder cancer, kidney cancer, ureter cancer, renal cell carcinoma, renal pelvis carcinoma, mesothelioma, hepatocellular cancer, biliary cancer, chronic leukemia, acute leukemia, lymphocytic lymphoma, CNS neoplasm, spinal axis cancer, brain stem glioma, glioblastoma multiform, astrocytoma, schwannoma, ependymoma, medulloblastoma, meningioma, squamous cell carcinoma and pituitary adenoma tumors, and tumor metastasis. In some embodiments, the targeting moiety specifically or preferentially binds to Her2/neu, and said cell is a cell from a breast cancer. In some embodiments, the targeting moiety specifically or preferentially binds to a primary antibody, and said primary antibody specifically binds to HER2/neu is a cell from a breast cancer.

In some embodiments, the targeting moiety specifically or preferentially binds to the Fc portion of an immunoglobulin (e.g., is a secondary antibody). For example, the targeting moiety may specifically or preferentially binds to the Fc portion of an IgG, an IgA, an IgD or IgM antibody. In some embodiments, the targeting moiety is selected from the group consisting of an antibody or antibody fragment, a unibody, an affybody, an aptamer, a ligand, and a polynucleotide. In some embodiments, the targeting moiety is an antibody or antibody fragment. In some embodiments, the targeting moiety is an antibody fragment selected from the group consisting of scFv, an Fv, an Fab, an Fab′, an F(ab)₂, a bis-scFv, heavy-light chains. In some embodiments, the targeting moiety is a monoclonal antibody. In some embodiments, the targeting moiety is a polyclonal antibody. In some embodiments, the antibody is a single domain antibody, a nanobody, a minibody, a diabody, a triabody, or a tetrabody. In some embodiments, the antibody is an IgG.

In some embodiments, the targeting moiety specifically or preferentially binds to a stem cell or a blood cell. For example, the targeting moiety can selectively or preferentially bind to a myeloid cell (e.g., monocytes and macrophages, neutrophils, basophils, eosinophils, erythrocytes, megakaryocytes/platelets, dendritic cells), or a lymphoid cell (e.g., T-cells, B-cells, NK-cells). In some embodiments, the targeting moiety specifically or preferentially binds to a stem cell biomarker selected from the group consisting of ABCG2, alpha 6, beta 1, B-catenin, C-myc, CK14, CK15, Ck19, CD34, CD71, CD117, CD133, Nestin, Oct-4, p63, p75 Neurotrophin R, NCAM, Sca-1, STRO-1.

In some embodiments, the detectable label is selected from the group consisting of a fluorescent label, an enzyme, a colorimetric label, a luminescent label, a radioactive label, a contrast agent, an MRI label, an electron spin label, and a magnetic label. In some embodiments, the detectable label comprises a fluorescent nanostructure. In some embodiments, the fluorescent nanostructure is selected from the group consisting of a quantum dot, a quantum rod and a quantum wire. In some embodiments, the detectable label comprises a radioactive label. In some embodiments, the radioactive label is selected from the group consisting of ³H, ¹²⁵I, ³⁵S, ¹⁴C, ³²P, ⁹⁹Tc, ²⁰³Pb, ⁶⁷Ga, ⁶⁸Ga, ⁷²As, ¹¹¹In, ^{113m}In, ⁹⁷Ru, ⁶²Cu, ⁶⁴Cu, ⁵²Fe, ^{52m}Mn, ⁵¹Cr, ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, and ¹¹¹Ag. In some embodiments, the radioactive label is attached via a chelator.

In a related aspect, the invention provides compositions comprising a population of nanoclusters or nanoaggregates, the preponderance of nanoclusters or nanoaggregates in said population comprising a plurality of crosslinked nanoparticles, said nanoparticles comprising a nanoscaffold core structure having attached thereto:

a targeting moiety comprising an antibody or antibody fragment; and

a detectable label;

wherein the nanoscaffold structure comprises a liposome and the average number or median number of nanoparticles in a nanocluster or nanoaggregate in said composition is about 2 or more. In some embodiments, the antibody specifically binds Her2/neu.

In another aspect, the present invention provides for a targeted nanocluster or nanoaggregate comprising: a crosslinked, nanoscaffold having attached thereto

(a) a primary or secondary moiety specific for a target, and

(b) at least two luminescent nanoparticles.

In another aspect, the present invention provides for a targeted nanocluster or nanoaggregate comprising: a crosslinked, nanoscaffold having attached thereto

(a) at least two primary or secondary antibody fragments specific for antigenic determinants of biological molecules or primary antibodies, and

(b) at least two luminescent nanoparticles.

In a further aspect, the invention provides methods of detecting the presence of and/or quantifying a biomarker, said method comprising:

a) contacting a subject or a biological sample suspected of containing the biomarker with a population of nanoclusters or nanoaggregates, as described herein; and

b) detecting the detectable label of the bound nanoclusters or nanoaggregates, whereby the presence of bound nanoclusters or nanoaggregates indicates the presence of and/or quantifies the biomarker.

In some embodiments, contacting a subject or a biological sample comprises administering the population of nanoclusters or nanoaggregates to the subject (i.e., the target biomarker is in vivo). In some embodiments, administering comprises administering the population of nanoclusters or nanoaggregates via a route selected from the group consisting of isophoretic delivery, transdermal delivery, aerosol administration, administration via inhalation, oral administration, intravenous administration, intraperitoneal administration and rectal administration. In various embodiments, the subject can be a human or a non-human mammal, for example, a non-human primate, a domesticated mammal (e.g., canine or feline), an agricultural mammal (e.g., equine, bovine, ovine, porcine), or a laboratory mammal (e.g., mouse, rat, rabbit, hamster). In some embodiments, detecting comprises using a detection modality selected from the group consisting of x-ray imaging, computerized axial tomography (CAT) scanning, magnetic resonance imaging (MRI), positron emission tomography (PET), electron spin resonance (ESR) detection, and thermographic imaging. In some embodiments, contacting a subject or a biological sample comprises contacting the population of nanoclusters or nanoaggregates to a biological sample. For example, the target biomarker is in vitro or ex vivo. In some embodiments, the biological sample comprises a sample selected from the group consisting of blood or a blood fraction, cerebrospinal fluid, urine, saliva, mucus, and a tissue sample. In some embodiments, the biological sample comprises a solid tissue sample or a cell suspension.

In some embodiments, the population of nanoclusters or nanoaggregates comprises a detection reagent formulated for use in an application selected from the group consisting of immunohistochemistry, immunocytochemistry, immunohistology, flow cytometry, ELISA, Western blot, dot blot, fluorescent in situ hybridization (FISH), high-resolution capillary isoelectric focusing, secondary ion mass spectrometry, mass cytometry and solid phase particle-based assays (e.g., microbead based assays, Luminex Bead Assays).

In some embodiments, detecting the presence of and/or quantifying a biomarker comprises detecting or quantifying a tumor or cancer cell. In some embodiments, detecting the presence of and/or quantifying a biomarker comprises detecting and/or quantifying a cancer marker selected from selected from Her2/neu, 5-alpha reductase, α-fetoprotein, AM-1, APC, APRIL, BAGE, β-catenin, Bc12, bcr-abl (b3a2), CA 125, CASP-8/FLICE, Cathepsins, CD19, CD20, CD21, CD23, CD22, CD38, CD33, CD35, CD44, CD45, CD46, CD5, CD52, CD55, CD59 (791Tgp72), CDC27, CDK4, CEA, c-myc, COX-2, Cytokeratin, DCC, DcR3, E6/E7, EGFR, EMBP, Ena78, Estrogen Receptor (ER), FGF8b and FGF8a, FLK 1/KDR, Folic Acid Receptor, G250, GAGE-Family, gastrin 17, Gastrin-releasing hormone (bombesin), GD2/GD3/GM2, GnRH, GnTV, gp100/Pme117, gp-100-in4, gp15, gp75/TRP-1, hCG, Heparanase, Her3, HMTV, Hsp70, hTERT (telomerase), IGFR1, IL 13R, iNOS, Ki 67, KIAA0205, K-ras, H-ras, N-ras, KSA (CO17-1A), LDLR-FUT, MAGE Family (MAGE1, MAGE3, etc.), Mammaglobin, MAP17, Melan-A/MART-1, mesothelin, MIC A/B, MT-MMP's, such as MMP2, MMP3, MMP7, MMP9, Mox1, Mucin, such as MUC-1, MUC-2, MUC-3, and MUC-4, MUM-1, NY-ESO-1, Osteonectin, p15, P170/MDR1, p53, p97/melanotransferrin, PAI-1, PDGF, Plasminogen (uPA), PRAME, Probasin, Progenipoietin, Progesterone Receptor (PR), PSA, PSM, RAGE-1, Rb, RCAS1, SART-1, SSX gene family, STAT3, STn (mucin assoc.), TAG-72, TGF-α, TGF-β, Thymosin β-15, IFN-γ, TPA, TPI, TRP-2, Tyrosinase, VEGF, ZAG, p16INK4, Glutathione and S-transferase. In some embodiments, detecting the presence of and/or quantifying a biomarker comprises detecting and/or quantifying Her2/neu.

In some embodiments, detecting and/or quantifying comprises detecting and/or quantifying a cell from a cancer selected from the group consisting of breast cancer, colorectal cancer, NSCLC, lung cancer, bone cancer, pancreatic cancer, skin cancer, cancer of the head or neck, cutaneous melanoma, intraocular melanoma, uterine cancer, ovarian cancer, rectal cancer, anal region cancer, stomach cancer, gastric cancer, colon cancer, breast cancer, uterine cancer, fallopian tube carcinoma, endometrial carcinoma, cervical carcinoma, vaginal carcinoma, vulval carcinoma, Hodgkin's Disease, esophagus cancer, small intestine cancer, endocrine system cancer, thyroid gland cancer, parathyroid gland cancer, adrenal gland cancer, soft tissue sarcoma, urethral cancer, penis cancer, prostate cancer, bladder cancer, kidney cancer, ureter cancer, renal cell carcinoma, renal pelvis carcinoma, mesothelioma, hepatocellular cancer, biliary cancer, chronic leukemia, acute leukemia, lymphocytic lymphoma, CNS neoplasm, spinal axis cancer, brain stem glioma, glioblastoma multiform, astrocytoma, schwannoma, ependymoma, medulloblastoma, meningioma, squamous cell carcinoma and pituitary adenoma tumors, and tumor metastasis.

A method of producing a population of nanoclusters or nanoaggregates, as described herein, said method comprising:

a) providing a nanoscaffold with at least a first functional group and a second functional group, wherein the first and second functional groups are different from each other and are suitable for crosslinking or conjugation;

b) attaching a targeting moiety to the first functional group;

c) attaching a detectable moiety to the second functional group;

wherein steps b) and c) can be performed in either order. In some embodiments, attaching is conjugating or crosslinking. In some embodiments, the detectable labels are associated with nanoscaffolds through other means, e.g., embedding, encapsulation, electrostatic interactions, chelation, binding pairs, (e.g., avidin-biotin binding).

In various embodiments, crosslinking between two or more nanoscaffolds occurs concurrently with either step b) or step c), thereby producing a population of nanoclusters or nanoaggregates. In some embodiments, the methods further comprise cross-linking multiple nanoscaffolds in a separate step, independent of steps b) and c), without affecting targeting moieties and detectable labels.

### DEFINITIONS

The term “nanoparticle” refers to a particle having a sub-micron (μm) size. In various embodiments, nanoparticles have a characteristic size (e.g., diameter) less than about 1 μm, 800 nm, or 500 nm, preferably less than about 400 nm, 300 nm, or 200 nm, more preferably about 100 nm or less, about 50 nm or less or about 30 or 20 nm or less.

The terms “nanocluster” or “nanoaggregate” interchangeably refer to an aggregation of two or more nanoscaffold core units. The two or more nanoscaffolds can be crosslinked to one another. A nanocluster or nanoaggregate may be comprised of 2, 3, 4, 5, 6, 7, 8, 9, 10, 20, 30, 50, 100, 150, 200, or more, nanoscaffold core units.

The term “nanoscaffold” refers to a nanoparticle structure concurrently attached to a multiplicity of targeting moieties and a multiplicity of detectable labels. Preferred nanoscaffold core units are less than about 100 nm, for example about 90 nm, 80 nm, 70 nm, 60 nm, 50 nm, 40 nm, 30 nm, 20 nm, or smaller. Illustrative nanoscaffolds can be comprised of a lipidic particle, a dendrimer, a hyperbranched polymer, a metal particle, a particle comprising a group II, III, or IV material, a polymeric nanoparticle, a glass nanoparticle, a quartz nanoparticle, a viral nanoparticle, a silicon oxide nanoparticle and a silica nanoparticle.

The term “lipidic particle” as used herein refers to amphipathic compounds which are capable of liposome formation, vesicle formation, micelle formation or emulsion formation.

The term “attached” refers to physical or chemical attachment, e.g., through covalent, ionic, electrostatic interactions, hydrophobic interaction, van der Waals force, hydrostatic or other means. “Attached to” includes without limitation surface conjugation, embedding, encapsulation, electrostatic interactions, chelation, binding via binding pairs (e.g., avidin-biotin binding)

The term “cancer markers” refers to biomolecules such as proteins that are useful in the diagnosis and prognosis of cancer. As used herein, “cancer markers” include but are not limited to: PSA, human chorionic gonadotropin, alpha-fetoprotein, carcinoembryonic antigen, cancer antigen (CA) 125, CA 15-3, CD20, CDH13, CD31, CD34, CD105, CD146, D16S422HER-2, phospatidylinositol 3-kinase (PI 3-kinase), trypsin, trypsin-1 complexed with alpha(1)-antitrypsin, estrogen receptor, progesterone receptor, c-erbB-2, be 1-2, S-phase fraction (SPF), p185erbB-2, low-affinity insulin like growth factor-binding protein, urinary tissue factor, vascular endothelial growth factor, epidermal growth factor, epidermal growth factor receptor, apoptosis proteins (p53, Ki67), factor VIII, adhesion proteins (CD-44, sialyl-TN, blood group A, bacterial lacZ, human placental alkaline phosphatase (ALP), alpha-difluoromethylornithine (DFMO), thymidine phosphorylase (dTHdPase), thrombomodulin, laminin receptor, fibronectin, anticyclins, anticyclin A, B, or E, proliferation associated nuclear antigen, lectin UEA-1, cea, 16, and von Willebrand's factor.

The terms “targeting moiety,” “ligand” or “binding moiety”, refer interchangeably to a molecule that binds to a particular target molecule and forms a bound complex as described above. The binding can be highly specific binding, however, in certain embodiments, the binding of an individual ligand to the target molecule can be with relatively low affinity and/or specificity. The ligand and its corresponding target molecule form a specific binding pair. Examples include, but are not limited to small organic molecules, sugars, lectins, nucleic acids, proteins, antibodies and fragments thereof, cytokines, receptor proteins, growth factors, nucleic acid binding proteins and the like which specifically bind desired target molecules, target collections of molecules, target receptors, target cells, and the like.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example Fab molecules can be displayed on phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No. 5,733,743). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). Particularly preferred antibodies should include all that have been displayed on phage (e.g., scFv, Fv, Fab and disulfide linked Fv (Reiter et al. (1995) Protein Eng. 8: 1323-1331). Antibody fragments that find use as targeting moieties include without limitation Fab′, F(ab′)₂, Fab, Fab₂, H+L (heavy chain+light chain), single domain antibodies, bivalent minibodies, scFv, bis-scFv, tascFv, bispecific Fab₂. See, Nelson, et al., Nature Biotechnology (2009) 27(4):331-337 and Holliger, et al., Nature Biotechnology (2005) 23(9):1126-1136.

The term “specifically binds”, as used herein, when referring to a targeting moiety or to a biomolecule (e.g., protein, nucleic acid, antibody, etc.), refers to a binding reaction that is determinative of the presence of the target molecule of the targeting moiety or biomolecule in a heterogeneous population of molecules (e.g., proteins and other biologics). Thus, under designated conditions (e.g., binding assay conditions in the case of a targeting moiety), the specified ligand or targeting moiety preferentially binds to its particular “target” molecule and preferentially does not bind in a significant amount to other molecules present in the sample.

An “effector” refers to any molecule or combination of molecules whose activity it is desired to deliver/into and/or localize at a target (e.g. at a cell displaying a characteristic marker). Effectors include, but are not limited to labels, cytotoxins, enzymes, growth factors, transcription factors, drugs, lipids, liposomes, etc.

The term “anti-cancer drug” or “anti-neoplastic agent” is used herein to refer to one or a combination of drugs conventionally used to treat cancer. Such drugs are well known to those of skill in the art and include, but are not limited to doxirubicin, vinblastine, vincristine, taxol, etc.

The term “immunoliposomes” refers to liposomes attached to an antibody or an antibody fragment and targeting capability.

A “reporter” is an effector that provides a detectable signal (e.g., is a detectable label or a detectable moiety). In certain embodiments, the reporter need not provide the detectable signal itself, but can simply provide a moiety that subsequently can bind to a detectable label.

The term “fluorescent nanostructure” refers to a nanoscale particle whose excitons are confined in all three spatial dimensions, as a result having properties that are between those of bulk materials and those of discrete molecules. A fluorescent nanostrucure is one where exciton confinement results in fluorescence.

The term “subject” refers to any mammal, including humans, non-human primates, domesticated mammals (e.g., canine or feline), agricultural mammals (e.g., equine, bovine, ovine, porcine), or laboratory mammals (e.g., mouse, rat, rabbit, hamster).

The term “plurality” refers to two or more.

The term “preponderance” refers to about 50% or more, for example, about 55%, 60%, 65%, 70%, 75% or more.

### BRIEF DESCRIPTION OF THE DRAWINGS

FIG. 1 illustrates a prior art method using traditional immunodetection (Prior Art)—conventional method of performing cell labeling.

FIG. 2 illustrates a prior art method using traditional immunodetection (Prior Art)—conventional method of performing an immunohistochemistry assay.

FIG. 3 illustrates exemplary targeted nanoclusters or nanoaggregates with primary or secondary antibody fragments applied to immunodetection.

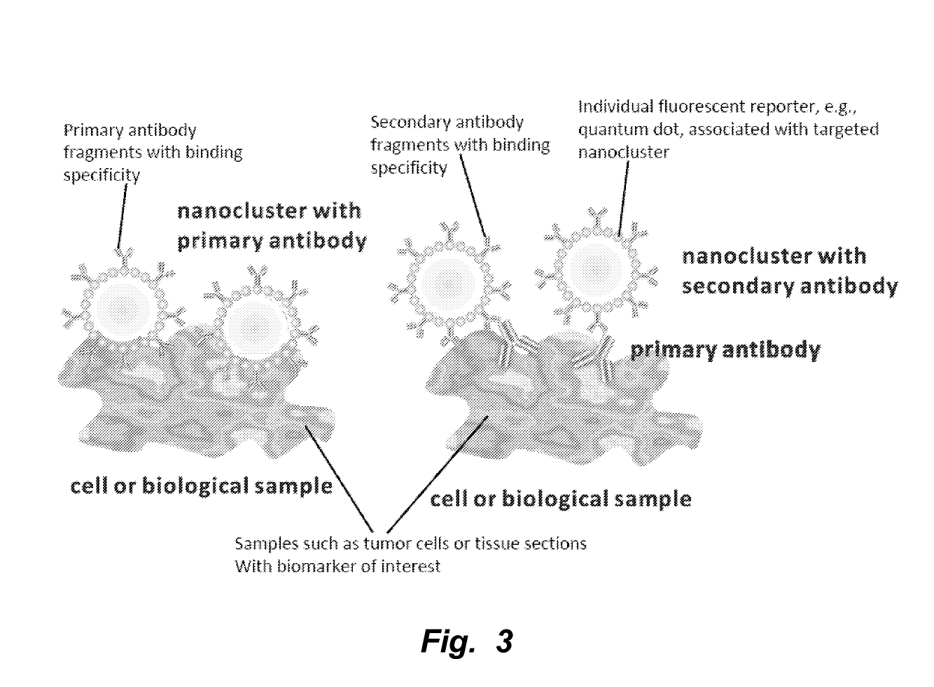

FIG. 4 illustrates exemplary configurations of crosslinked nanoclusters or nanoaggregates. One, two, three, or more core units are chemically linked into one cluster consisting of multiplicity of nanoscaffolds, antigen binding components, and optical or fluorescent reporters.

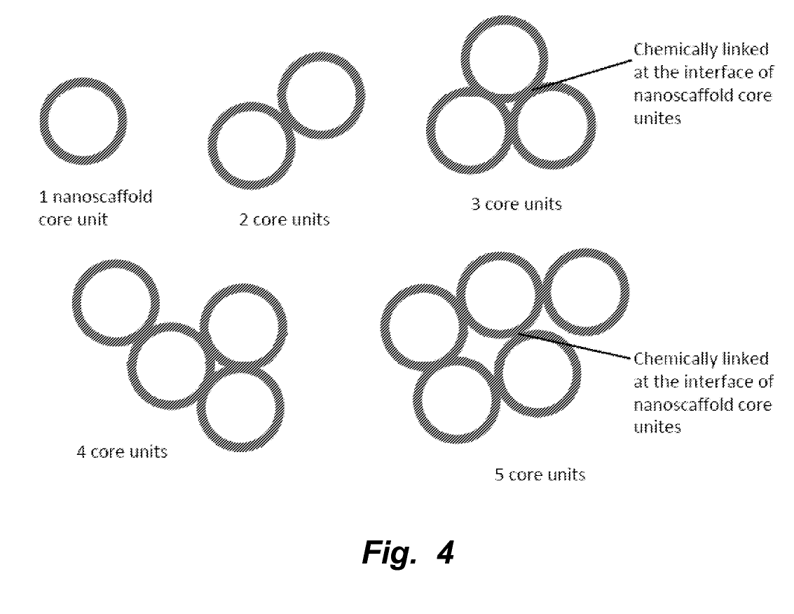

FIGS. 5A-B. Cryo-electron microscopy (cryoEM) images showing examples of crosslinked nanoclusters or nanoaggregates.

FIG. 6A-B. Flow cytometry analysis of human epidermal growth factor receptor 2 (HER2/erbB2) in human breast cancer cells MDA-MB-453 and MDA-MB-468. A. Control sample of mixed MDA-MB-453 and MDA-MB-468 cells. Cells emerged as one population in the FL2 channel histogram of flow cytometer. B. Mixed MDA-MB-453 and MDA-MB-468 cells incubated with mouse anti-HER2 monoclonal antibody and goat anti-mouse nanocluster. Cells emerged as two populations in the FL2 channel histogram of flow cytometer. M1 region corresponds to MDA-MB-453 population and M2 region corresponds to MDA-MB-468 population.

FIG. 7. Mean fluorescence intensities from flow cytometry analysis of human epidermal growth factor receptor 2 (HER2/erbB2) in human breast cancer cells MDA-MB-453 and MCF-7. Comparison of signals from cells labeled by commercially available Qdot IgG conjugate (quantum dots directly conjugated to antibody) and Qdot nanocluster or nanoaggregate is shown. The results indicate signal amplification by Qdot nanocluster.

FIG. 8. Fluorescence microscopy images for SK-BR-3 cells using the targeted nanocluster.

FIG. 9. Fluorescence microscopy images for MCF-7 cells using the targeted nanocluster. Panels from left to right: DAPI staining for cell nucleus; fluorescence emission at 605 nm by 405 nm excitation, indicating distribution of Qdot 605 targeted nanoclusters or nanoaggregates; and the merged images.

FIG. 10. Fluorescence Microscopy Images for MDA-MB-468 cells using the targeted nanocluster. Together with SK-BR-3 and MCF-7, the results validated targeted nanoclusters or nanoaggregates across the range of erbB2 expression from high to negative cell lines, and can be used to establish reference standards for comparison and staining results.

### DETAILED DESCRIPTION

### 1. Introduction

Multifunctional nanoparticles are a versatile platform for cancer diagnosis and treatment. Nanoparticles that carry multiple modalities and functionalities in targeting, reporting and drug delivery, find use in oncology and other medical applications.¹⁶ The present invention is based, in part, on the design of multifunctional nanoparticles, in particular, targeted nanoclusters or nanoaggregates, that combine targeting and reporting capabilities for immunodetection, and providing higher sensitivity, greater dynamic range, multiplex reporting, and more quantitative results can be achieved with simplified procedures.

### 2. Polyvalent Nanoscaffolds

The backbones of the presently described targeted nanoclusters or nanoaggregates are nanoscale macromolecular assemblies, e.g., of an average diameter of about 500 nm or less, for example, 400 nm, 300 nm, 200 nm, 100 nm, or less, for example, 90 nm, 80 nm, 70 nm, 60 nm, 50 nm, or less. The nanoscaffolds described herein provide flexibility for chemical modification and functionalization that result in both a multiplicity and a defined ratio of specific functional groups. The functional groups on the nanoscaffolds can be selectively and efficiently conjugated to various targeted biologic molecules. The nanoscaffolds further can withstand any chemical process needed to modify the functional groups while remaining biologically inert.

In one embodiment, the nanoscaffolds comprise lipids. Lipids are amphiphilic molecules that self-assemble to form micelles or vesicles under certain conditions in aqueous environments. Lipid-based vesicles present one such versatile platform that can be controlled precisely their compositions, functionalities, and sizes. An advantage of using lipid vesicles as nanoscaffolds is the functionality can be installed before synthesis and assembly of the micellar or vesicular platform. Therefore, no chemical derivatization is needed for implementing the functional groups for bioconjugation. Furthermore, the targeting moiety (e.g., antigen binding moiety, antibody or antibody fragment) can be pre-conjugated to functionalized lipid and inserted into pre-formed liposomes above the transition temperature of the lipid layers,^{17, 18} thus eliminating the necessity of post-modification and conjugation. To further increase the number of functional components associated with lipid nanoscaffolds, multiple units of lipid cores can be chemically linked at their interfaces. The crosslinking is confined to the extent that the resulting nanoclusters or nanoaggregates are still in a homogeneous phase, do not precipitate out of the suspension, thus achieving crosslinked configurations.

### Lipidic Particles

In certain embodiments, the nanoparticles are lipidic particles. Lipidic particles are nanoparticles that include at least one lipid component forming a condensed lipid phase. Typically, a lipidic particle has preponderance of lipids in its composition. The exemplary condensed lipid phases are solid amorphous or true crystalline phases; isomorphic liquid phases (droplets); and various hydrated mesomorphic oriented lipid phases such as liquid crystalline and pseudocrystalline bilayer phases (L-alpha, L-beta, P-beta, Lc), interdigitated bilayer phases, and nonlamellar phases (inverted hexagonal H-I, H-II, cubic Pn3m) (see The Structure of Biological Membranes, ed. by P. Yeagle, CRC Press, Bora Raton, Fla., 1991, in particular ch. 1-5, incorporated herein by reference.) Lipidic particles include, but are not limited to a liposome, a lipid-nucleic acid complex, a lipid-drug complex, a solid lipid particle, and a microemulsion droplet. Methods of making and using these types of lipidic particles, as well as attachment of affinity moieties, e.g., antibodies, to them are known in the art (see, e.g., U.S. Pat. Nos. 5,077,057; 5,100,591; 5,616,334; 6,406,713 (drug-lipid complexes); U.S. Pat. Nos. 5,576,016; 6,248,363; Bondi et al., Drug Delivery vol. 10, p. 245-250, 2003; Pedersen et al., Eur. J. Pharm. Biopharm. v. 62, p. 155-162, 2006 (solid lipid particles); U.S. Pat. Nos. 5,534,502; 6,720,001; Shiokawa et al., Clin. Cancer Res. v. 11, p. 2018-2025, 2005 (microemulsions); U.S. Pat. No. 6,071,533 (lipid-nucleic acid complexes)).

### Liposomes and Lipid Vesicles

A liposome is generally defined as a particle comprising one or more lipid bilayers enclosing an interior, typically an aqueous interior. Thus, a liposome is often a vesicle formed by a bilayer lipid membrane. There are many methods for the preparation of liposomes. Some of them are used to prepare small vesicles (d<0.05 micrometer), some for larger vesicles (d>0.05 micrometer). Some are used to prepare multilamellar vesicles, some for unilamellar ones. For the present invention, unilamellar vesicles are preferred because a lytic event on the membrane means the lysis of the entire vesicle. However, multilamellar vesicles could also be used, perhaps with reduced efficiency. Methods for liposome preparation are exhaustively described in several review articles such as Szoka and Papahadjopoulos (1980) Ann. Rev. Biophys. Bioeng., 9: 467, Deamer and Uster (1983) Pp. 27-51 In: Liposomes, ed. M. J. Ostro, Marcel Dekker, New York, and the like.

In various embodiments, liposomes of the invention are composed of vesicle-forming lipids, generally including amphipathic lipids having both hydrophobic tail groups and polar head groups. A characteristic of a vesicle-forming lipid is its ability to either (a) form spontaneously into bilayer vesicles in water, as exemplified by the phospholipids, or (b) be stably incorporated into lipid bilayers, by having the hydrophobic portion in contact with the interior, hydrophobic region of the bilayer membrane, and the polar head group oriented toward the exterior, polar surface of the membrane. A vesicle-forming lipid for use in the present invention is any conventional lipid possessing one of the characteristics described above.

In certain embodiments the vesicle-forming lipids of this type are preferably those having two hydrocarbon tails or chains, typically acyl groups, and a polar head group. Included in this class are the phospholipids, such as phosphatidylcholine (PC), phosphatidylethanolamine (PE), phosphatidic acid (PA), phosphatidylglycerol (PG), and phosphatidylinositol (PI), where the two hydrocarbon chains are typically between about 14-22 carbon atoms in length, and have varying degrees of unsaturation. In certain embodiments preferred phospholipids include PE and PC. One illustrative PC is hydrogenated soy phosphatidylcholine (HSPC). Single chain lipids, such as sphingomyelin (SM), and the like can also be used.

The above-described lipids and phospholipids whose acyl chains have a variety of degrees of saturation can be obtained commercially, or prepared according to published methods. Other lipids that can be included in certain embodiments are sphingolipids and glycolipids. The term “sphingolipid” as used herein encompasses lipids having two hydrocarbon chains, one of which is the hydrocarbon chain of sphingosine. The term “glycolipids” refers to shingolipids comprising also one or more sugar residues.

Lipids for use in the lipidic particles described herein can include relatively “fluid” lipids, meaning that the lipid phase has a relatively low lipid melting temperature, e.g., at or below room temperature, or alternately, relatively “rigid” lipids, meaning that the lipid has a relatively high melting point, e.g., at temperatures up to 50° C. As a general rule, the more rigid, i.e., saturated lipids, contribute to greater membrane rigidity in the lipid bilayer structure, and thus to more stable drug retention after active drug loading. In certain embodiments preferred lipids of this type are those having phase transition temperatures above about 37° C.

In various embodiments the liposomes may additionally include lipids that can stabilize a vesicle or liposome composed predominantly of phospholipids. An illustrative lipids of this group is cholesterol at levels between 25 to 45 mole percent.

In certain embodiments liposomes used in the invention contain between 30 to 75 percent phospholipids, e.g., phosphatidylcholine (PC), 25-45 percent cholesterol. One illustrative liposome formulation contains about 60-66 mole percent, e.g., about 60, 61, 62, 63, 64, 65, 66 mole percent, phosphatidylcholine, and about 34-40 mole percent, e.g., about 34, 35, 36, 37, 38, 39 or 40 mole percent, cholesterol.

In various embodiments the liposomes of the invention include a surface coating of a hydrophilic polymer chain. “Surface-coating” refers to the coating of any hydrophilic polymer on the surface of liposomes. The hydrophilic polymer is included in the liposome by including in the liposome composition one or more vesicle-forming lipids derivatized with a hydrophilic polymer chain. The vesicle-forming lipids which can be used are any of those described above for the first vesicle-forming lipid component, however, in certain embodiments, vesicle-forming lipids with diacyl chains, such as phospholipids, are preferred. One illustrative phospholipid is phosphatidylethanolamine (PE), which contains a reactive amino group convenient for coupling to the activated polymers. One illustrative PE is distearoyl PE (DSPE). Another example is non-phospholipid double chain amphiphilic lipids, such as diacyl- or dialkylglycerols, derivatized with a hydrophilic polymer chain.

In certain embodiments a hydrophilic polymer for use in coupling to a vesicle forming lipid is polyethyleneglycol (PEG), preferably as a PEG chain having a molecular weight between 1,000-10,000 Daltons, more preferably between 1,000-5,000 Daltons, most preferably between 2,000-5,000 Daltons. Methoxy or ethoxy-capped analogues of PEG are also useful hydrophilic polymers, commercially available in a variety of polymer sizes, e.g., 120-20,000 Daltons.

Other hydrophilic polymers that can be suitable include, but are not limited to polylactic acid, polyglycolic acid, polyvinylpyrrolidone, polymethyloxazoline, polyethyloxazoline, polyhydroxypropyl methacrylamide, polymethacrylamide, polydimethylacrylamide, and derivatized celluloses, such as hydroxymethylcellulose or hydroxyethylcellulose.

Preparation of lipid-polymer conjugates containing these polymers attached to a suitable lipid, such as PE, have been described, for example in U.S. Pat. No. 5,395,619, which is expressly incorporated herein by reference, and by Zalipsky in STEALTH LIPOSOMES (1995). In certain embodiments, typically, between about 1-20 mole percent of the polymer-derivatized lipid is included in the liposome-forming components during liposome formation. Polymer-derivatized lipids suitable for practicing the invention are also commercially available (e.g. SUNBRITE(R), NOF Corporation, Japan, and Avanti Polar Lipids, Alabama, USA).

In various embodiments the hydrophilic polymer chains provide a surface coating of hydrophilic chains sufficient to extend the blood circulation time of the liposomes in the absence of such a coating. The extent of enhancement of blood circulation time is severalfold over that achieved in the absence of the polymer coating, as described in U.S. Pat. No. 5,013,556, which is expressly incorporated herein by reference.

The liposomes may be prepared by a variety of techniques, including those detailed in Szoka et al. (1980) Ann. Rev. Biophys. Bioeng. 9: 467. In certain embodiments the liposomes are multilamellar vesicles (MLVs). MLVs can be formed by simple lipid-film hydration techniques. In an illustrative procedure, a mixture of liposome-forming lipids and including a vesicle-forming lipid derivatized with a hydrophilic polymer are dissolved in a suitable organic solvent which is evaporated in a vessel to form a dried thin film. The film is then covered by an aqueous medium to form MLVs, typically with sizes between about 0.1 to 10 microns. Further illustrative methods of preparing derivatized lipids and of forming polymer-coated liposomes have been described in U.S. Pat. Nos. 5,013,556, 5,631,018 and 5,395,619, which are incorporated herein by reference.

After liposome formation, the vesicles may be sized to achieve a size distribution of liposomes within a selected range, according to known methods. In certain embodiments the liposomes are uniformly sized to a selected size range between 0.04 to 0.25 μm. Small unilamellar vesicles (SUVs), typically in the 0.04 to 0.08 μm range, can be prepared by extensive sonication or homogenization of the liposomes. Homogeneously sized liposomes having sizes in a selected range between about 0.08 to 0.4 microns can be produced, e.g., by extrusion through polycarbonate membranes or other defined pore size membranes having selected uniform pore sizes ranging from 0.03 to 0.5 microns, typically, 0.05, 0.08, 0.1, or 0.2 microns. The sizing is typically carried out in the original lipid-hydrating buffer, so that the liposome interior spaces retain this medium throughout the initial liposome processing steps.

In certain embodiments the liposomes are prepared to include an ion gradient, such as a pH gradient or an ammonium or amine ion gradient, across the liposome lipid bilayerin order to effect loading of the liposomes with a substance of interest, e.g., a pharmaceutical (drug). A liposome may also contain substances, such as polyvalent ions, reducing the rate of drug escape from the liposome. One method for preparing such liposomes loaded with a drug is set forth in U.S. Patent Publication 2007/0116753 which is incorporated herein by reference.

In one illustrative approach a mixture of liposome-forming lipids is dissolved in a suitable organic solvent and evaporated in a vessel to form a thin film. The film is then covered with an aqueous medium containing the solute species that will form the aqueous phase in the liposome interior spaces in the final liposome preparation. The lipid film hydrates to form multi-lamellar vesicles (MLVs), typically with heterogeneous sizes between about 0.1 to 10 microns. The liposome are then sized, as described above, to a uniform selected size range.

After sizing, the external medium of the liposomes can be treated to produce an ion gradient across the liposome membrane, which is typically a lower inside/higher outside concentration gradient. This may be done in a variety of ways, e.g., by (i) diluting the external medium, (ii) dialysis against the desired final medium, (iii) molecular-sieve chromatography, e.g., using SEPHADEX G-50, against the desired medium, or (iv) high-speed centrifugation and resuspension of pelleted liposomes in the desired final medium. The external medium which is selected can depend on the mechanism of gradient formation and the external pH desired.

The liposomes can be loaded with a therapeutic moiety, e.g., an anticancer agent or an antineoplastic agent. Any method known in the art for loading the desired therapeutic agent can be used. In one approach, a proton gradient is used for drug loading, e.g., by creating an ammonium ion gradient across the liposome membrane, as described, for example, in U.S. Pat. No. 5,192,549. Further methods that find use for loading of therapeutic agents into the central space of a liposome are described in, e.g., Drummond, et al., “Intraliposomal Trapping Agents for Improving In Vivo Liposomal Drug Formulation Stability,” in Liposome Technology, 3rd ed.; Gregoriadis, G., Ed. Informa Healthcare USA: 2007; Vol. II, pp 149-168; Drummond, et al., Journal of Pharmaceutical Sciences (2008) 97(11):4696-4740; Drummond, et al., Journal of Pharmacology And Experimental Therapeutics (2009) 328(1):321-330; Noble, et al., Cancer Chemotherapy and Pharmacology (2009) 64(4):741-51, all incorporated herein by reference.

While the foregoing discussion pertains to the formation of liposomes, similar lipids and lipid compositions can be used to form other lipidic particles such as a solid lipid particle, a microemulsion, and the like.

Methods of functionalizing lipids and liposomes with affinity moieties such as antibodies are well known to those of skill in the art (see, e.g., DE 3,218,121; Epstein et al. (1985) Proc. Natl. Acad. Sci., USA, 82:3688 (1985); Hwang et al. (1980) Proc. Natl. Acad. Sci., USA, 77: 4030; EP 52,322; EP 36,676; EP 88,046; EP 143,949; EP 142,641; Japanese patent application 83-118008; U.S. Pat. Nos. 4,485,045 and 4,544,545; and EP 102,324, all of which are incorporated herein by reference). One preferred method for attachment of proteinaceous affinity moieties to lipidic microparticles, applicable to lipidic particles, is described in U.S. Pat. No. 6,210,707, incorporated herein by reference.

Another kind of nanoparticle suitable for practicing the instant invention is a micelle. As used herein, a “micelle” refers to an aggregate of amphiphilic molecules in an aqueous medium, having an interior core and an exterior surface, wherein the amphiphilic molecules are predominantly oriented with their hydrophobic portions forming the core and hydrophilic portions forming the exterior surface. Micelles are typically in a dynamic equilibrium with the amphiphilic molecules or ions from which they are formed existing in solution in a non-aggregated form. Many amphiphilic compounds, including in particular. detergents, surfactants, amphiphilic polymers, lipopolymers (such as PEG-lipids), bile salts, single-chain phospholipids and other single-chain amphiphiles, and amphipathic pharmaceutical compounds are known to spontaneously form micelles in aqueous media above certain concentration, known as critical micellization concentration, or CMC. Unlike lipidic particles, amphipathic, e.g., lipid, components of a micelle, as defined herein, do not form bilayer phases, nonbilayer mesophases, isotropic liquid phases or solid amorphous or crystalline phases. The concept of a micelle, as well as the methods and conditions for their formation, are well known to skilled in the art. micelles can co-exist in solution with lipidic particles. See, for example, Liposome Technology, Third Edition, vol. 1, ch. 11, p. 209-239, Informa, London, 2007. Micelles are useful in carryring and targeting pharmaceutical agents. The uses of micelles as carriers for pharmaceuticals as well as the methods of making pharmaceutical micelles and attachment to micelles of moieties having affinity to target cells and/or tissues, including affinity moieties binding to EGFR, are known in the art (see, e.g., Torchilin (2007) Pharmaceutical Res. 24: 1-16; Lukyanov and Torchilin (2004) Adv. Drug Delivery Reviews 56: 1273-1289; Torchilin et al. (2003) Proc. Natl. Acad. Sci., USA, 100: 6039-6044; Zeng et al. (2006) Bioconjugate Chemistry 17: 399-409; Sutton et al. (2007) Pharmaceutical Research 24: 1029-1046; Lee et al. (2007) Molecular Pharmacology, 4: 769-781, all incorporated herein by reference).

An illustrative lipid particle core comprises 60 mol % 1,2-distearoyl-sn-glycero-3-phosphocholine (DSPC), 37.5 mol % cholesterol, 2 mol % 1,2-distearoyl-sn-glycero-3-phosphoethanolamine-N-[amino(polyethylene glycol)2000] (amine-PEG2000-DSPE), and 0.5 mol % 1,2-distearoyl-sn-glycero-3-phosphoethanolamine-N-[maleimide(polyethylene glycol)2000] (maleimide-PEG2000-DSPE), but is not limited to such.

Nanoscale lipidic particles, including lipid micelles, lipid vesicles and multilamellar vesicles, can be formed by any method known in the art. Methods for producing lipid vesicles are described, e.g., in Sternberg, B., Freeze-Fracture Electron Microscopy of Liposomes. In Liposome Technology 2nd Edition Volume I Liposome Preparation and Related Techniques, 2nd ed.; Gregoriadis, G., Ed. CRC Press: Boca Raton, Ann Arbor, London, Tokyo, 1993; Vol. 1, pp 363-383; Düzgüne, N.; Gregoriadis, G., Introduction: The Origins of Liposomes: Alec Bangham at Babraham. Methods in Enzymology 2005, 391, 1-3; and Gregoriadis, G., Liposome Technology. Third Edition ed.; Informa Healthcare USA, Inc.: New York, 2007, all of which are hereby incorporated by reference in their entirety for all purposes.

An illustrative method for preparing lipid vesicles is by extrusion. A typical procedure for extrusion is as follows: Dry lipid mixture is prepared by lyophilization or evaporation. An extrusion apparatus is temperature-controlled by, for example, water bath circulation or a heating block on a hot plate. Hydrate lipid mixture using a suitable buffer for >30 min. The lipid suspension should be kept above the phase transition temperature of the lipid during hydration and extrusion. To increase the efficiency of entrapment of water-soluble compounds, one may subject the hydrated lipid suspension to a few freeze/thaw cycles by alternately placing the sample vial in a dry ice bath and warm water bath. Once the sample is fully hydrated, load the sample into one of the extruder. Allow the temperature of the lipid suspension to equilibrate with the temperature of the extruder. Apply pressure until the lipid solution is completely transferred through the porous membranes. Repeat the above procedure for a total of 10 passes or more through membrane. In general, the more passes though the membrane, the more homogenous the vesicle solution becomes. For vesicles without organic dyes, the vesicle suspension should begin to clarify to yield a slightly hazy transparent solution. The haze is due to light scattering induced by residual large particles remaining in the suspension. These particles can be removed by centrifugation to yield a clear suspension of small unilamellar vesicles. Collect the vesicle solution from the extruder into a clean sample vial. When not in use, store the vesicle solution at 4° C., preferably without freezing. Storage of vesicle solutions is preferably in physiological buffers of pH 7; at higher temperatures and pH<5 or >8 may reduce the lifetime of the vesicle suspension.

Another illustrative method for lipid vesicle preparation is by sonication. A typical procedure for sonication is as follows: Dry lipid mixture is prepared by evaporation followed by lyophilization. Hydrate lipid mixture using a suitable buffer for >30 min. The lipid suspension should be kept above the phase transition temperature of the lipid during hydration. One may subject the hydrated lipid suspension to a few freeze/thaw cycles by alternately placing the sample vial in a dry ice bath and warm water bath. Once the sample is fully hydrated, subject the sample to sonication. Sonication can be generated by a sonicator probe, a sonicator bath, or equivalent apparatus. The resulting vesicle solution typically has a broader size distribution. The properties are similar to that prepared by extrusion. Storage requirements are the same as above.

### Polymers

In another embodiment, the nanoscaffold comprises a polymer, including organic or inorganic polymers, branched or unbranched. Such polymers can include but are not limited to, organic polymers, inorganic polymers, amphiphilic polymers, hyperbranched polymers, sugars, carbohydrates, polysaccharides, nucleotides, DNA, or RNA. Multiple nanoscaffold cores can be crosslinked to further increase the number of functional components.

Hyperbranched polymers and dendrimers also provide polyvalent nanoscaffolds for conjugation of multiple targeting moieties and optical labels. Forming nanoclusters with cross-linked hyperbranched polymers or dendrimers as nanoparticle core is feasible.

### Hyperbranched Polymers

In another embodiment, the nanoscaffold can comprise a hyper-branched polymer. In another embodiment, the nanoscaffold comprise a hyperbranched polymer. Hyperbranched polymers are another class of versatile nanoparticles in that their size, functionality, chemical and physical properties can be controlled. In one embodiment, the polymer is an amphiphilic hyperbranched polymer that is capable of forming micelle-like structure and encapsulate hydrophobic nanoparticles such as uncoated quantum dots. The hyperbranched polymer can be an “imperfect” molecule, in that it may include linear sections, and may feature random or unsymmetrical branching. A hyperbranched polymer is a less complex structure synthesized in a single step reaction from functional monomers, or polycondensation, ring-opening multibranched polymerization, self-condensing vinyl polymerization, etc. Hyperbranched polymers can be selectively modified to achieve multiple functionalities on the surface and linked to functional components such as carbon chains to install hydrophobicity, and primary amine groups for hydrophilicity and activation for subsequent modifications.

The advantages of hyperbranched polymers include smaller unit sizes (typically <60 nm in diameter) and relatively simple procedures for synthesis. Potential disadvantages include broad size distributions and difficult control of surface modification for specific functionalities. Preparation of hyperbranched polymers, e.g., hyperbranched polyglycerols, is well documented and typically performed as follows: Controlled anionic ring-opening multibranching polymerization of glycidol is performed to form hyperbranched polyglycerols (Sunder, et al., Macromolecules (1999) 32(13):4240-4246: Kainthan, et al., Biomacromolecules (2006) 7(3):703-709). Hyperbranched polyglycerols are then reacted with succinic anhydride in pyridine to install carboxylic acid terminal groups via an ester linkage (Haxton, et al., Dalton Transactions (2008) (43):5872-5875). Carbon-13 NMR can be used to characterize the presence and ratio of terminal carboxylic acid groups. Once the functional group content on hyperbranched polyglycerols is verified, hydroxyl is further functionalized by the following scheme: hyperbranched polyglycerols-OH+N-(p-maleimidophenyl)isocyanate (PMPI, 10-fold molar excess) in DMSO or DMF at pH 8.5 to obtain hyperbranched polyglycerols-maleimide. Hyperbranched polyglycerols thus possess both carboxyl and maleimide functional groups that can react with corresponding cross-linkers and chemical groups, or can be further derivatized to suit specific functional groups available.

### Dendrimers

In one embodiment, the nanoscaffold comprises a dendrimer. A dendrimer is a branched polymer structure, preferably a synthetic polymer structure. Substantially the entire molecule is branched and the size of the dendrimer is controlled. The dendrimer can feature functional groups for the attachment of the targeting moiety and the luminescent nanoparticle elements of the nanocluster. In addition, a dendrimer can include a multi-functional core, repeated branching units, surface functional groups, and can be synthesized in a multi-step process.

Dendrimers find applications in clinical oncology and biomedical research (Tekade, et al., Chemical Reviews (2009) 109(1):49-87; Svenson, et al., Advanced Drug Delivery Reviews (2005) 57(15):2106-2129; Lee, et al., Nature Biotechnology (2005) 23(12):1517-1526). Conjugation with antibodies for diagnostic or therapeutic purposes was also reported (Wangler, et al., Bioconjugate Chemistry (2008) 19(4):813-820). Forming nanoclusters based on dendrimer cores possibly further enhances the intended function. The advantages of dendrimers include well defined globular structure and unit size, a large number of attachment points on the periphery of nanoparticle, increased solubility compared to their linear analogues, controllable steric crowding that creates an interior capable of encapsulating small molecules, etc. The potential disadvantages include relatively complicated synthesis procedures, and difficult control of surface modification for installation of specific functional groups. Preparation of dendrimers is well documented and is typically performed via either divergent or convergent approaches (Bosman, et al., Chemical Reviews (1999) 99(7):1665-1688; Kojima, et al., Bioconjugate Chemistry (2000) 11(6):910-917; Dykes, Journal of Chemical Technology & Biotechnology (2001) 76(9):903-918; Grayson, et al., Chemical Reviews (2001) 101(12):3819-3868). Depending on desired properties, building blocks, and synthetic approach used, a wide variety of dendrimers can be synthesized, such as poly(amidoamine) (PAMAM), polypropylene imine) (PPI), dendritic poly(amides), poly(esters), poly-(urethanes), poly(carbonates), poly(aryl ethers), poly(arylamines), poly(aryl ketones), poly(aryl alkynes), poly(aryl methanes), poly(arylammonium) salts, poly(thioureas), poly-(ether imides), poly(keto ethers), poly(amine ethers), poly(amino esters), poly(amide ethers), poly(pyridyl amides), poly(uracils), poly(triazenes), poly(saccharides), poly(glycopeptides), and poly(nucleic acids), etc. Surface derivatization for specific functional groups can be performed similar to functionalization of hyperbranched polymers described above.

Dynamic light scattering or gel permeation chromatography can be used to estimate the size and molecular weight of nanoclusters consisting of hyperbranched polymer or dendrimer nanoscaffolds. Sample purity can be evaluated by matrix-assisted laser desorption ionization time-of-flight mass spectroscopy (MALDI-TOF MS)

Periodic Table Group II, III, IV, V, or VI materials

Group II, III, IV, V, or VI materials (e.g., Group II, III, IV, V, or VI elements, semiconductors, and/or oxides thereof), more preferably to essentially any or all Group III, IV, or V materials (e.g., carbon, silicon, germanium, tin, lead), doped Group II, III, IV, V, and VI elements, or oxides of pure or doped Group II, III, IV, V, or VI elements also find use as a nanoscaffold. In certain preferred embodiments, the nanoscaffold is a Group III, IV, or V material, more preferably a Group IV material (oxide, and/or doped variant), still more preferably silicon or germanium or a doped and/or oxidized silicon or germanium.

The Group II, III, IV, V, or VI element can be essentially pure, or it can be doped (e.g., p- or n-doped). P- and n-dopants for use with Group II-VI elements, in particular for use with Groups III, IV, and V elements, more particularly for use with Group IV elements (e.g., silicon, germanium, etc.) are well known to those of skill in the art. Such dopants include, but are not limited to phosphorous compounds, boron compounds, arsenic compounds, aluminum compounds, and the like. Many doped Group II, III, IV, V, or VI elements are semiconductors and include, but are not limited to ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AlS, AlP, AlSb, PbS, PbSe, Ge and Si and ternary and quaternary mixtures thereof.

### Other Materials for Use as Nanoscaffolds

Other illustrative nanoscaffold structures that find use include without limitation, carbon nanotubes, Bucky balls, metal and metal oxides particles (including magnetic particles), silicon oxides (silica) particles, glass particles, quartz particles, polymer micelles, plastic nanobeads, and virus particles and capsids. Illustrative viruses include retroviruses (including lentiviruses), picornaviruses, flaviviruses, pox viruses, herpes viruses, potiviruses, and other plant and animal viruses.

### 3. Targeting Moiety

In one embodiment, the targeted nanocluster or nanoaggregate further comprises a targeting moiety which is specific for an antigen or a ligand. In one embodiment, there is at least one targeting moiety. In another embodiment, there are at least two targeting moieties for detection. The multiple targeting moieties can be directed to the same antigen or to different antigens.

Such targeting moieties can be any moiety that has a specific binding partner including but not limited to, primary or secondary antibodies, antibody fragments, antigen binding molecules, oligonucleotides, aptamers, probes, carbohydrates, sugars, proteins, enzymes, peptides, small molecules, or drugs. In one embodiment, oligonucleotide ligands for hybridization to a specific identifying sequence are the targeting moiety. In another embodiment, the targeting moiety is a secondary antibody having affinity for a primary antibody. And in yet another embodiment, the targeting moiety is an affinity compound such as a small molecule which binds to specific target.

Targets Associated with Hyperproliferation Disorders

In various embodiments, the targeting moiety is a molecule that specifically or preferentially binds a marker expressed by (e.g., on the surface of) or associated with the target cell(s). While essentially any cell can be targeted, certain preferred cells include those associated with a pathology characterized by hyperproliferation of a cell (i.e., a hyperproliferative disorder). Illustrative hyperproliferative disorders include, but are not limited to psoriasis, neutrophilia, polycythemia, thrombocytosis, and cancer.

Hyperproliferative disorders characterized as cancer include but are not limited to solid tumors, such as cancers of the breast, respiratory tract, brain, reproductive organs, digestive tract, urinary tract, eye, liver, skin, head and neck, thyroid, parathyroid and their distant metastases. These disorders also include lymphomas, sarcomas, and leukemias. Examples of breast cancer include, but are not limited to invasive ductal carcinoma, invasive lobular carcinoma, ductal carcinoma in situ, and lobular carcinoma in situ. Examples of cancers of the respiratory tract include, but are not limited to small-cell and non-small-cell lung carcinoma, as well as bronchial adenoma and pleuropulmonary blastoma. Examples of brain cancers include, but are not limited to brain stem and hypothalmic glioma, cerebellar and cerebral astrocytoma, medulloblastoma, ependymoma, as well as neuroectodermal and pineal tumor. Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus. Tumors of the digestive tract include, but are not limited to anal, colon, colorectal, esophageal, gallbladder, gastric, pancreatic, rectal, small-intestine, and salivary gland cancers. Tumors of the urinary tract include, but are not limited to bladder, penile, kidney, renal pelvis, ureter, and urethral cancers. Eye cancers include, but are not limited to intraocular melanoma and retinoblastoma. Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma. Skin cancers include, but are not limited to squamous cell carcinoma, Kaposi's sarcoma, malignant melanoma, Merkel cell skin cancer, and non-melanoma skin cancer. Head-and-neck cancers include, but are not limited to laryngeal/hypopharyngeal/nasopharyngeal/oropharyngeal cancer, and lip and oral cavity cancer. Lymphomas include, but are not limited to AIDS-related lymphoma, non-Hodgkin's lymphoma, cutaneous T-cell lymphoma, Hodgkin's disease, and lymphoma of the central nervous system. Sarcomas include, but are not limited to sarcoma of the soft tissue, osteosarcoma, malignant fibrous histiocytoma, lymphosarcoma, and rhabdomyosarcoma. Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering the nanocluster or nanoaggregate compositions.

In certain embodiments, the targeting moiety is a moiety that binds a cancer marker (e.g., a tumor associated antigen). A wide variety of cancer markers are known to those of skill in the art. The markers need not be unique to cancer cells, but can also be effective where the expression of the marker is elevated in a cancer cell (as compared to normal healthy cells) or where the marker is not present at comparable levels in surrounding tissues (especially where the chimeric moiety is delivered locally).

Illustrative cancer markers include, for example, the tumor marker recognized by the ND4 monoclonal antibody. This marker is found on poorly differentiated colorectal cancer, as well as gastrointestinal neuroendocrine tumors (see, e.g., Tobi et al. (1998) Cancer Detection and Prevention, 22(2): 147-152). Other important targets for cancer immunotherapy are membrane bound complement regulatory glycoprotein: CD46, CD55 and CD59, which have been found to be expressed on most tumor cells in vivo and in vitro. Human mucins (e.g. MUC1) are known tumor markers as are gp100, tyrosinase, and MAGE, which are found in melanoma. Wild-type Wilms' tumor gene WT1 is expressed at high levels not only in most of acute myelocytic, acute lymphocytic, and chronic myelocytic leukemia, but also in various types of solid tumors including lung cancer.

Acute lymphocytic leukemia has been characterized by the TAAs HLA-Dr, CD1, CD2, CD5, CD7, CD19, and CD20. Acute myelogenous leukemia has been characterized by the TAAs HLA-Dr, CD7, CD13, CD14, CD15, CD33, and CD34. Breast cancer has been characterized by the markers EGFR, HER2, MUC1, Tag-72. Various carcinomas have been characterized by the markers MUC1, TAG-72, and CEA. Chronic lymphocytic leukemia has been characterized by the markers CD3, CD19, CD20, CD21, CD25, and HLA-DR. Hairy cell leukemia has been characterized by the markers CD19, CD20, CD21, CD25. Hodgkin's disease has been characterized by the Leu-M1 marker. Various melanomas have been characterized by the HMB 45 marker. Non-hodgkins lymphomas have been characterized by the CD20, CD19, and Ia marker. And various prostate cancers have been characterized by the PSMA and SE10 markers.

In addition, many kinds of tumor cells display unusual antigens that are either inappropriate for the cell type and/or its environment, or are only normally present during the organisms' development (e.g. fetal antigens). Examples of such antigens include the glycosphingolipid GD2, a disialoganglioside that is normally only expressed at a significant level on the outer surface membranes of neuronal cells, where its exposure to the immune system is limited by the blood-brain barrier. GD2 is expressed on the surfaces of a wide range of tumor cells including neuroblastoma, medulloblastomas, astrocytomas, melanomas, small-cell lung cancer, osteosarcomas and other soft tissue sarcomas. GD2 is thus a convenient tumor-specific target for immunotherapies.

Other kinds of tumor cells display cell surface receptors that are rare or absent on the surfaces of healthy cells, and which are responsible for activating cellular signaling pathways that cause the unregulated growth and division of the tumor cell. Examples include (ErbB2). HER2/neu, a constitutively active cell surface receptor that is produced at abnormally high levels on the surface of breast cancer tumor cells.

Other useful targets include, but are not limited to CD20, CD52, CD33, epidermal growth factor receptor and the like.

An illustrative, but not limiting list of suitable tumor markers is provided in Table 1. Antibodies to these and other cancer markers are known to those of skill in the art and can be obtained commercially or readily produced, e.g. using phage-display technology.

TABLE 1
Table 1. Illustrative cancer markers and associated references, all of which are
incorporated herein by reference for the purpose of identifying the referenced tumor
markers.

| Marker | Reference |
|---|---|
| 5 alpha reductase | Délos et al. (1998) Int J Cancer, 75: 6 840-846 |
| α-fetoprotein | Esteban et al. (1996) Tumour Biol., 17(5): 299-305 |
| AM-1 | Harada et al. (1996) Tohoku J Exp Med., 180(3): 273-288 |
| APC | Dihlmannet al. (1997) Oncol Res., 9(3) 119-127 |
| APRIL | Sordat et al. (′998) J Exp Med., 188(6): 1185-1190 |
| BAGE | Böel et al. (1995) Immunity, 2: 167-175. |
| β-catenin | Hugh et al. (1999) Int J Cancer, 82(4): 504-11 |
| Bc12 | Koty et al. (1999) Lung Cancer, 23(2): 115-127 |
| bcr-abl (b3a2) | Verfaillie et al.(′996) Blood, 87(11): 4770-4779 |
| CA-125 | Bast et al. (′998) Int J Biol Markers, 13(4): 179-187 |
| CASP-8/FLICE | Mandruzzato et al. (1997) J Exp Med., 186(5): 785-793. |
| Cathepsins | Thomssen et al.(1995) Clin Cancer Res., 1(7): 741-746 |
| CD19 | Scheuermann et al. (1995) Leuk Lymphoma, 18(5-6): 385-397 |
| CD20 | Knox et al. (1996) Clin Cancer Res., 2(3): 457-470 |
| CD21, CD23 | Shubinsky et al. (1997) Leuk Lymphoma, 25(5-6): 521-530 |
| CD22, CD38 | French et al. (1995) Br J Cancer, 71(5): 986-994 |
| CD33 | Nakase et al. (1996) Am J Clin Pathol., 105(6): 761-768 |
| CD35 | Yamakawa et al. Cancer, 73(11): 2808-2817 |
| CD44 | Naot et al. (1997) Adv Cancer Res., 71: 241-319 |
| CD45 | Buzzi et al. (1992) Cancer Res., 52(14): 4027-4035 |
| CD46 | Yamakawa et al. (1994) Cancer, 73(11): 2808-2817 |
| CD5 | Stein et al. (1991) Clin Exp Immunol., 85(3): 418-423 |
| CD52 | Ginaldi et al. (1998) Leuk Res., 22(2): 185-191 |
| CD55 | Spendlove et al. (1999) Cancer Res., 59: 2282-2286. |
| CD59 (791Tgp72) | Jarvis et al. (1997) Int J Cancer, 71(6): 1049-1055 |
| CDC27 | Wang et al. (1999) Science, 284(5418): 1351-1354 |
| CDK4 | Wolfel et al. (1995) Science, 269(5228): 1281-1284 |
| CEA | Kass et al. (1999) Cancer Res., 59(3): 676-683 |
| c-myc | Watson et al. (1991) Cancer Res., 51(15): 3996-4000 |
| Cox-2 | Tsujii et al. (1998) Cell, 93: 705-716 |
| DCC | Gotley et al. (1996) Oncogene, 13(4): 787-795 |
| DcR3 | Pitti et al. (1998) Nature, 396: 699-703 |
| E6/E7 | Steller et al. (1996) Cancer Res., 56(21): 5087-5091 |
| EGFR | Yang et al. (1999) Cancer Res., 59(6): 1236-1243. |
| EMBP | Shiina et al. (1996) Prostate, 29(3): 169-176. |
| Ena78 | Arenberg et al. (1998) J. Clin. Invest., 102: 465-472. |
| FGF8b and FGF8a | Dorkin et al. (1999) Oncogene, 18(17): 2755-2761 |
| FLK-1/KDR | Annie and Fong (1999) Cancer Res., 59: 99-106 |
| Folic Acid Receptor | Dixon et al. (1992) J Biol Chem., 267(33): 24140-72414 |
| G250 | Divgi et al. (1998) Clin Cancer Res., 4(11): 2729-2739 |
| GAGE-Family | De Backer et al. (1999) Cancer Res., 59(13): 3157-3165 |
| gastrin 17 | Watson et al. (1995) Int J Cancer, 61(2): 233-240 |
| Gastrin-releasing | Wang et al. (1996) Int J Cancer, 68(4): 528-534 |
| hormone (bombesin) | |
| GD2/GD3/GM2 | Wiesner and Sweeley (1995) Int J Cancer, 60(3): 294-299 |
| GnRH | Bahk et al. (1998) Urol Res., 26(4): 259-264 |
| GnTV | Hengstler et al. (1998) Recent Results Cancer Res., 154: 47-85 |
| gp100/Pmel17 | Wagner et al. (1997) Cancer Immunol Immunother., 44(4): 239-247 |
| gp-100-in4 | Kirkin et al. (1998) APMIS, 106(7): 665-679 |
| gp15 | Maeurer et al.(1996) Melanoma Res., 6(1): 11-24 |
| gp75/TRP-1 | Lewis et al.(1995) Semin Cancer Biol., 6(6): 321-327 |
| hCG | Hoermann et al. (1992) Cancer Res., 52(6): 1520-1524 |
| Heparanase | Vlodavsky et al. (1999) Nat Med., 5(7): 793-802 |
| Her2/neu | Lewis et al. (1995) Semin Cancer Biol., 6(6): 321-327 |
| Her3 | |
| HMTV | Kahl et al.(1991) Br J Cancer, 63(4): 534-540 |
| Hsp70 | Jaattela et al. (1998) EMBO J., 17(21): 6124-6134 |
| hTERT | Vonderheide et al. (1999) Immunity, 10: 673-679. 1999. |
| (telomerase) | |
| IGFR1 | Ellis et al. (1998) Breast Cancer Res. Treat., 52: 175-184 |
| IL-13R | Murata et al. (1997) Biochem Biophys Res Commun., 238(1): 90-94 |
| iNOS | Klotz et al. (1998) Cancer, 82(10): 1897-1903 |
| Ki 67 | Gerdes et al. (1983) Int J Cancer, 31: 13-20 |
| KIAA0205 | Guéguen et al. (1998) J Immunol., 160(12): 6188-6194 |
| K-ras, H-ras, | Abrams et al. (1996) Semin Oncol., 23(1): 118-134 |
| N-ras | |
| KSA | Zhang et al. (1998) Clin Cancer Res., 4(2): 295-302 |
| (CO17-1A) | |
| LDLR-FUT | Caruso et al. (1998) Oncol Rep., 5(4): 927-930 |
| MAGE Family | Marchand et al. (1999) Int J Cancer, 80(2): 219-230 |
| (MAGE1, | |
| MAGE3, etc.) | |
| Mammaglobin | Watson et al. (1999) Cancer Res., 59: 13 3028-3031 |
| MAP17 | Kocher et al. (1996) Am J Pathol., 149(2): 493-500 |
| Melan-A/ | Lewis and Houghton (1995) Semin Cancer Biol., 6(6): 321-327 |
| MART-1 | |
| mesothelin | Chang et al. (1996) Proc. Natl. Acad. Sci., USA, 93(1): 136-140 |
| MIC A/B | Groh et al.(1998) Science, 279: 1737-1740 |
| MT-MMP's, such as | Sato and Seiki (1996) J Biochem (Tokyo), 119(2): 209-215 |
| MMP2, MMP3, | |
| MMP7, MMP9 | |
| Mox1 | Candia et al. (1992) Development, 116(4): 1123-1136 |
| Mucin, such as MUC- | Lewis and Houghton (1995) Semin Cancer Biol., 6(6): 321-327 |
| 1, MUC-2, MUC-3, | |
| and MUC-4 | |
| MUM-1 | Kirkin et al. (1998) APMIS, 106(7): 665-679 |
| NY-ESO-1 | Jager et al. (1998) J. Exp. Med., 187: 265-270 |
| Osteonectin | Graham et al. (1997) Eur J Cancer, 33(10): 1654-1660 |
| p15 | Yoshida et al. (1995) Cancer Res., 55(13): 2756-2760 |
| P170/MDR1 | Trock et al. (1997) J Natl Cancer Inst., 89(13): 917-931 |
| p53 | Roth et al. (1996) Proc. Natl. Acad. Sci., USA, 93(10): 4781-4786. |
| p97/melanotransferrin | Furukawa et al. (1989) J Exp Med., 169(2): 585-590 |
| PAI-1 | Grøndahl-Hansen et al. (1993) Cancer Res., 53(11): 2513-2521 |
| PDGF | Vassbotn et al. (1993) Mol Cell Biol., 13(7): 4066-4076 |
| Plasminogen (uPA) | Naitoh et al. (1995) Jpn J Cancer Res., 86(1): 48-56 |
| PRAME | Kirkin et al. (1998) APMIS, 106(7): 665-679 |
| Probasin | Matuo et al. (1985) Biochem Biophys Res Commun., 130(1): 293-300 |
| Progenipoietin | — |
| PSA | Sanda et al. (1999) Urology, 53(2): 260-266. |
| PSM | Kawakami et al.(1997) Cancer Res., 57(12): 2321-2324 |
| RAGE-1 | Gaugler et al.(1996) Immunogenetics, 44(5): 323-330 |
| Rb | Dosaka-Akita et al. (1997) Cancer, 79(7): 1329-1337 |
| RCAS1 | Sonoda et al.(1996) Cancer, 77(8): 1501-1509. |
| SART-1 | Kikuchi et al.(1999( Int J Cancer, 81(3): 459-466 |
| SSX gene | Gure et al. (1997) Int J Cancer, 72(6): 965-971 |
| family | |
| STAT3 | Bromberg et al. (1999) Cell, 98(3): 295-303 |
| STn | Sandmaier et al. (1999) J Immunother., 22(1): 54-66 |
| (mucin assoc.) | |
| TAG-72 | Kuroki et al. (1990) Cancer Res., 50(16): 4872-4879 |
| TGF-α | Imanishi et al. (1989) Br J Cancer, 59(5): 761-765 |
| TGF-β | Picon et al. (1998) Cancer Epidemiol Biomarkers Prey, 7(6): 497-504 |
| Thymosin β 15 | Bao et al. (1996) Nature Medicine. 2(12), 1322-1328 |
| IFN-α | Moradi et al. (1993) Cancer, 72(8): 2433-2440 |
| TPA | Maulard et al. (1994) Cancer, 73(2): 394-398 |
| TPI | Nishida et al.(1984) Cancer Res 44(8): 3324-9 |
| TRP-2 | Parkhurst et al. (1998) Cancer Res., 58(21) 4895-4901 |
| Tyrosinase | Kirkin et al. (1998) APMIS, 106(7): 665-679 |
| VEGF | Hyodo et al. (1998) Eur J Cancer, 34(13): 2041-2045 |
| ZAG | Sanchez et al. (1999) Science, 283(5409): 1914-1919 |
| p16INK4 | Quelle et al. (1995) Oncogene Aug. 17, 1995; 11(4): 635-645 |
| Glutathione | Hengstler (1998) et al. Recent Results Cancer Res., 154: 47-85 |
| S-transferase | |

Any of the foregoing markers can be used as targets for the targeting moieties comprising the interferon-targeting moiety constructs of this invention. In certain embodiments the target markers include, but are not limited to members of the epidermal growth factor family (e.g., HER2, HER3, EGF, HER4), CD1, CD2, CD3, CD5, CD7, CD13, CD14, CD15, CD19, CD20, CD21, CD23, CD25, CD33, CD34, CD38, 5E10, CEA, HLA-DR, HM 1.24, HMB 45, 1a, Leu-M1, MUC1, PMSA, TAG-72, phosphatidyl serine antigen, and the like.

The foregoing markers are intended to be illustrative and not limiting. Other tumor associated antigens will be known to those of skill in the art. Moreover, some of the CD markers listed in Table 1 also find use for stem cell identification and selection, and white blood cell characterization.

Where the tumor marker is a cell surface receptor, ligand to that receptor can function as targeting moieties. Similarly mimetics of such ligands can also be used as targeting moieties.

In certain embodiments, the targeting moieties can comprise antibodies, unibodies, or affybodies that specifically or preferentially bind the tumor marker. Antibodies that specifically or preferentially bind tumor markers are well known to those of skill in the art. Thus, for example, antibodies that bind the CD22 antigen expressed on human B cells include HD6, RFB4, UV22-2, Tol5, 4 KB128, a humanized anti-CD22 antibody (hLL2) (see, e.g., Li et al. (1989) Cell. Immunol. 111: 85-99; Mason et al. (1987) Blood 69: 836-40; Behr et al. (1999) Clin. Cancer Res. 5: 3304s-3314s; Bonardi et al. (1993) Cancer Res. 53: 3015-3021).

Antibodies to CD33 include for example, HuM195 (see, e.g., Kossman et al. (1999) Clin. Cancer Res. 5: 2748-2755), CMA-676 (see, e.g., Sievers et al., (1999) Blood 93: 3678-3684.

Antibodies to CD38 include for example, AT13/5 (see, e.g., Ellis et al. (1995) J. Immunol. 155: 925-937), HB7, and the like.

In certain embodiments the targeting moiety comprises an anti-HER2 antibody. The ergB 2 gene, more commonly known as (Her-2/neu), is an oncogene encoding a transmembrane receptor. Several antibodies have been developed against Her-2/neu, including trastuzumab (e.g., HERCEPTIN®.; Formier et al. (1999) Oncology (Huntingt) 13: 647-58), TAB-250 (Rosenblum et al. (1999) Clin. Cancer Res. 5: 865-874), BACH-250 (Id.), TA1 (Maier et al. (1991) Cancer Res. 51: 5361-5369), and the mAbs described in U.S. Pat. Nos. 5,772,997; 5,770,195 (mAb 4D5; ATCC CRL 10463); and U.S. Pat. No. 5,677,171

Illustrative anti-MUC-1 antibodies include, but are not limited to Mc5 (see, e.g., Peterson et al. (1997) Cancer Res. 57: 1103-1108; Ozzello et al. (1993) Breast Cancer Res. Treat. 25: 265-276), and hCTMO1 (see, e.g., Van H of et al. (1996) Cancer Res. 56: 5179-5185).

Illustrative anti-TAG-72 antibodies include, but are not limited to CC49 (see, e.g., Pavlinkova et al. (1999) Clin. Cancer Res. 5: 2613-2619), B72.3 (see, e.g., Divgi et al. (1994) Nucl. Med. Biol. 21: 9-15), and those disclosed in U.S. Pat. No. 5,976,531.

Illustrative anti-HM1.24 antibodies include, but are not limited to a mouse monoclonal anti-HM1.24 IgG2a/κ and a a humanized anti-HM1.24 IgG1/κ. antibody (see, e.g., Ono et al. (1999) Mol. Immuno. 36: 387-395).

A number of antibodies have been developed that specifically bind HER2 and some are in clinical use. These include, for example, trastuzumab (e.g., HERCEPTIN®, Formier et al. (1999) Oncology (Huntingt) 13: 647-658), TAB-250 (Rosenblum et al. (1999) Clin. Cancer Res. 5: 865-874), BACH-250 (Id.), TA1 (see, e.g., Maier et al. (1991) Cancer Res. 51: 5361-5369), and the antibodies described in U.S. Pat. Nos. 5,772,997; 5,770,195, and 5,677,171.

Other fully human anti-HER2/neu antibodies are well known to those of skill in the art. Such antibodies include, but are not limited to the C6 antibodies such as C6.5, DPL5, G98A, C6 MH3-B1, B1D2, C6VLB, C6VLD, C6VLE, C6VLF, C6 MH3-D7, C6 MH3-D6, C6 MH3-D5, C6 MH3-D3, C6 MH3-D2, C6 MH3-D1, C6 MH3-C4, C6 MH3-C3, C6 MH3-B9, C6 MH3-B5, C6 MH3-B48, C6 MH3-B47, C6 MH3-B46, C6 MH3-B43, C6 MH3-B41, C6 MH3-B39, C6 MH3-B34, C6 MH3-B33, C6 MH3-B31, C6 MH3-B27, C6 MH3-B25, C6 MH3-B21, C6 MH3-B20, C6 MH3-B2, C6 MH3-B16, C6 MH3-B15, C6 MH3-B11, C6 MH3-B1, C6 MH3-A3, C6 MH3-A2, and C6ML3-9. These and other anti-HER2/neu antibodies are described in U.S. Pat. Nos. 6,512,097 and 5,977,322, in PCT Publication WO 97/00271, in Schier et al. (1996) J Mol Biol 255: 28-43, Schier et al. (1996) J Mol Biol 263: 551-567, and the like.

More generally, antibodies directed to various members of the epidermal growth factor receptor family are well suited for use as targeting moieties in the present nanoclusters or nanoaggregates. Such antibodies include, but are not limited to anti-EGF-R antibodies as described in U.S. Pat. Nos. 5,844,093 and 5,558,864, and in European Patent No. 706,799A). Other illustrative anti-EGFR family antibodies include, but are not limited to antibodies such as C6.5, C6ML3-9, C6 MH3-B1, C6-B1D2, F5, HER3.A5, HER3.F4, HER3.H1, HER3.H3, HER3.E12, HER3.B12, EGFR.E12, EGFR.C10, EGFR.B11, EGFR.E8, HER4.B4, HER4.G4, HER4.F4, HER4.A8, HER4.B6, HER4.D4, HER4.D7, HER4.D11, HER4.D12, HER4.E3, HER4.E7, HER4.F8 and HER4.C7 and the like (see, e.g., U.S. Patent publications US 2006/0099205 A1 and US 2004/0071696 A1 which are incorporated herein by reference).

As described in U.S. Pat. Nos. 6,512,097 and 5,977,322 other anti-EGFR family member antibodies can readily be produced by shuffling light and/or heavy chains followed by one or more rounds of affinity selection. Thus in certain embodiments, this invention contemplates the use of one, two, or three CDRs in the VL and/or VH region that are CDRs described in the above-identified antibodies and/or the above identified publications.

In various embodiments the targeting moiety comprises an antibody that specifically or preferentially binds CD20. Anti-CD20 antibodies are well known to those of skill and include, but are not limited to RITUXIMAB®, Ibritumomab tiuxetan, and tositumomab, AME-133v (Applied Molecular Evolution), Ocrelizumab (Roche), Ofatumumab (Genmab), TRU-015 (Trubion) and IMMU-106 (Immunomedics).

In various embodiments, the targeting moiety specifically or preferentially binds to caspase-3 or caspase-9, enzymes involved in apoptosis and implicated in cancer. Detection can be accomplished using any applicable assay format known in the art, including flow cytometry.

### Stem Cells Biomarker Targets

In various embodiments, the targeting moiety specifically or preferentially binds to a biomarker for stem cells detection. Stem cell surface biomarkers for cancer stem cells, embryonic stem cells, mesenchymal stem cells, neuronal stem cells, endothelial progenitors, hematopoietic progenitors, lineage markers for endoderm, ectoderm and mesoderm, and signaling pathways are known in the art. Antibodies against stem cell surface biomarkers are commercially available and provided by, e.g., Abcam (Cambridge, Mass. and on the internet at abcam.com). Stem cell biomarker targets of interest include, without limitation ABCG2, alpha 6, beta 1, B-catenin, C-myc, CK14, CK15, Ck19, CD34, CD71, CD117, CD133, Nestin, Oct-4, p63, p75 Neurotrophin R, NCAM, Sca-1 and STRO-1.

### Hematological Biomarker Targets

In various embodiments, the targeting moiety specifically or preferentially binds to a biomarker for a desired subset of blood cells. Surface biomarkers for specific blood cells including lymphocytes (e.g., T cells and B cells), antigen presenting cells, macrophages, mast cells, neutrophils, eosinophils, NK cells, myeloid cells, among others, are known in the art. Antibodies against blood cell surface biomarkers are commercially available and provided by, e.g., Abcam (Cambridge, Mass. and on the internet at abcam.com). Illustrative lymphocyte biomarker targets that find use include CD3, CD4 (T cells), CD7, CD8 (T cells), CD10, CD19 (NK cells, B cells), CD20, CD45RO, CD45RA, CD56 (NK cells, B cells), Bc12 and Bc16. Illustrative myeloma biomarkers that find use include CD38 and CD138. Other useful target proteins associated with myeloma are summarized in Rawstron, et al., Haematologia (2008) 93(3):431-438. Such targets can be detected using the nanoclusters in any applicable detection assay known in the art, including flow cytometry. Quantum dot-labeled antibodies against several blood cell biomarkers are commercially available, e.g., human CD2 (clone S5.5), human CD3 (clones UCHT1 and S4.1), human CD4 (clone S3.5), human CD8 (clone 3B5), human CD10 (clone MEM-78), human CD14 (clone TüK4), human CD19 (clone SJ25-C1), human CD20 (clone HI47), human CD27 (clone CLB-27/1), human CD38 (clone HIT2), human CD45 (clone HI30), human CD45RA (clone MEM-56), human CD56 (clone MEM-188), human HLA-DR (clone TU36), mouse CD3 (clone 145-2C11), mouse CD4 (clone RM4-5), mouse CD19 (clone 6D5), mouse CD45R (B220) (clone RA3-6B2). The antibody clones also find use as targeting moieties attached to the present nanoclusters or nanoaggregates.

### Attaching the Targeting Moiety to the Nanoscaffold

The targeting moiety can be attached covalently or non-covalently, reversibly or non-reversibly to the nanoscaffold. Generally, the targeting moiety is attached to the nanoscaffold through a functionalized group. In other embodiments, the targeting moiety can be adsorbed onto the surface.

In one embodiment, the targeting moiety is attached to the nanoscaffold through a crosslinker or spacer, which are known in the art. Crosslinkers having polyethylene glycol (PEG), also referred to as polyethyleneoxide (PEO), or spacers are convenient alternatives to reagents with purely hydrocarbon spacer arms. Moreover, PEG spacers improve water solubility of reagent and conjugate, reduce the potential for aggregation of the conjugate, and increases flexibility of the crosslink, resulting in reduced immunogenic response to the spacer itself. By contrast to typical PEG reagents that contain heterogeneous mixtures of different PEG chain lengths, these PEO reagents are homogeneous compounds of defined molecular weight and spacer arm length, providing greater precision in optimization and characterization of crosslinking applications. For example, in one embodiment, the sulfhydryl groups in the primary or secondary antibody are reduced to allow attachment to the nanoscaffold surface. In one example, reagents including maleimide, disulfide and the process of acylation can be used to form a direct covalent bond with a cysteine on the nanoscaffold surface.

In general, any affinity molecule useful in the prior art, in combination with a known ligand to provide specific recognition of a detectable substance will find utility in the attachment of the targeted moiety to the nanoscaffold. Examples of such biological molecules which can then be attached to these functional groups include linker molecules having a known binding partner, or affinity molecule, include but are not limited to, polysaccharides, lectins, selectins, nucleic acids (both monomeric and oligomeric), proteins, enzymes, lipids, folic acid (folate), antibodies, and small molecules such as sugars, peptides, aptamers, drugs, and ligands.

In another embodiment, the attachment is covalent. A bifunctional crosslinker useful for the invention would comprise two different reactive groups capable of coupling to two different functional targets such as peptides, proteins, macromolecules, semiconductor nanocrystals, or substrate. The two reactive groups can be the same or different and include but are not limited to such reactive groups as thiol, carboxylate, carbonyl, amine, hydroxyl, aldehyde, ketone, active hydrogen, ester, sulfhydryl or photoreactive moieties. For examples, in one embodiment, a crosslinker can have one amine-reactive group and a thiol-reactive group on the functional ends. Further examples of heterobifunctional crosslinkers that may be used as linking agents in the invention include but are not limited to:

amine reactive+sulfhydryl-reactive crosslinkers.

amine reactive+carbonyl-reactive crosslinkers.

carbonyl-reactive+sulfhydryl-reactive crosslinkers.

amine-reactive+photoreactive crosslinkers

sulfhydryl-reactive+photoreactive crosslinkers

carbonyl-reactive+photoreactive crosslinkers.

carboxylate-reactive+photoreactive crosslinkers

arginine-reactive+photoreactive crosslinkers

Below is a list of categories in which crosslinkers generally fit. The list is illustrative and should not be considered exhaustive of the types of crosslinkers that may be useful for the invention. For each category, i.e. which functional group these chemicals target, there are some subcategories, because one reactive group is capable of reacting with several functional groups.

Most crosslinkers with reactive groups can be broadly classified in the following categories:

TABLE 2
Crosslinkers

| | |
|---|---|
| Amine-reactive | the crosslinker coupled to a amine (NH₂) |
| | containing molecule |
| Thiol-reactive | the crosslinker couple to a sulfhydryl (SH) |
| | containing molecule |
| Carboxylate-reactive | the crosslinker coupled to a carboxylic acid |
| | (COOH) containing molecule |
| Hydroxyl-reactive | the crosslinker coupled to a hydroxyls (—OH) |
| | containing molecule |
| Aldehyde- and ketone- | the crosslinker coupled to an aldehyde |
| reactive | (—CHO) or ketone (R₂CO) containing |
| | molecule |
| Active hydrogen-reactive | the crosslinker that contains activatable |
| | hydrogen or reacts with an active hydrogen |
| Photo-reactive | the crosslinker that is activated or reacts with a |
| | chemical group activated by light, such as |
| | benzophenone |

More specifically, chemicals entering in these categories include, but are not limited to those containing:

TABLE 3
Functional Groups

| | |
|---|---|
| 1 | Isothiocyanates, isocyanates, acyl azides, NHS esters, sulfonyl |
| | chlorides, aldehydes and glyoxals, epoxides and oxiranes, |
| | carbonates, arylating agents, imidoesters, carbodiimides, |
| | anhydrides, alkynes |
| 2 | Haloacetyl and alkyl halide derivates, maleimides, aziridines, acryloyl |
| | derivatives, arylating agents, thiol-disulfides exchange reagents |
| 3 | Diazoalkanes and diazoacetyl compounds, such as |
| | carbonyldiimidazoles and carbodiimides |
| 4 | Epoxides and oxiranes, carbonyldiimidazole, oxidation with |
| | periodate, N,N′-disuccinimidyl carbonate or N-hydroxylsuccimidyl |
| | chloroformate, enzymatic oxidation, alkyl halogens, isocyanates |
| 5 | Hydrazine derivatives for schiff base formation or reduction |
| | amination |
| 6 | Diazonium derivatives for mannich condensation and |
| | iodination reactions |
| 7 | Aryl azides and halogenated aryl azides, benzophenones, diazo |
| | compounds, diazirine derivatives |

For each of these subcategories there are many examples of chemicals. All these chemicals and the above list of subcategories are described in the prior art, but many can be found in, “Bioconjugate Techniques” by Greg T Hermanson, Academic Press, San Diego, 2008, which is hereby incorporated by reference.

In one embodiment, the targeting moiety is an antibody attached to the nanoscaffold, i.e., a primary antibody. The antibody can be against any antigen or target of interest, as described herein

In some embodiments, the antibody is a secondary antibody, i.e., an antibody that binds to a primary antibody bound to a target antigen. Secondary antibodies can be derived from the same sources and methods as primary antibodies. They bind to primary antibodies or antibody fragments against which they are raised. Illustrative secondary antibodies include combinations of A anti-B from the following combinations: Cow, Dog, Goat, Horse, Lama, Mouse, Rabbit, Rat, Sheep, Swine, wherein the listed animals can either provide the source or the target of the secondary antibody. The fragmentation of anti-mouse secondary antibody was achieved by multiple trials of various fragmentation techniques. Typically, IgG antibody consists of multiple components that can be digested and reduced. Various chemical agents and conditions exist for this purpose, yet results differ for different antibodies

For example, goat anti-mouse secondary antibody, goat anti-rabbit secondary antibody, rabbit anti-horse secondary antibody, etc. The choice of specific combination depends on the primary antibody one uses, the type of specimen, and the method of detection. For example, in order to obtain consistent and comparable results for erbB2 immunostaining on formalin-fixed, paraffin-embedded patient biopsy tissue sections, a suitable antibody to use can be mouse monoclonal IgG—Clone TAB250, IgG1-kappa isotype, which is derived from raising antibodies against immunogen NIH3T3 cells transfected with the c-erbB-2 gene. To apply secondary amplification for this particular primary antibody, an anti-mouse secondary antibody is used. The species of origin for the secondary antibody also has to do with affinity, specificity, and background blocking

Fragmentation of IgG is often used to generate specific functional groups for site-directed conjugation and functionalization of certain substrates. Several reducing agents including 2-mercaptoethanol (2-ME), 2-mercaptoethylamine (2-MEA), and dithiothreitol (DTT)¹⁹ are often used for this purpose. Subjecting IgG to these reducing agents result in various mixtures of antibody fragments. Some fragments do not contain the antigen-binding moieties and others could be “over-reduced” and lose the antigen-binding capability and rendered inactive. It is often empirical to optimize the conditions for specific antibody to yield desired fragments. In one example, goat anti-mouse secondary antibody was reduced by adding 100 μl of 50 mM 2-ME, 2-MEA, or DTT to 400 μl of goal anti-mouse secondary antibody and incubate at 37° C. for 30 minutes. The mixture was transferred to ice water bath and desalted by spin columns immediately afterwards.

Antibodies include polyclonal antibodies, monoclonal antibodies, synthetic antibodies, antibodies, or immunogenically active fragments, or derivatives, thereof. Exemplary fragments are F(ab′)2, Fab′, Fab, Fv, scFv, bis-scFv, heavy-light chains, and the like. In various embodiments, the targeting moiety may be a single domain antibody, a nanobody, a minibody, a diabody, a triabody, or a tetrabody.

The invention need not be limited to the use of the antibodies described above, and other such antibodies as they are known to those of skill in the art can be used in the compositions and methods described herein. It will be recognized that affybodies, unibodies, and other antigen binding molecules can be used instead of antibodies.

UniBody refers to antibody technology that produces a stable, smaller antibody format with an anticipated longer therapeutic window than certain small antibody formats. In certain embodiments unibodies are produced from IgG4 antibodies by eliminating the hinge region of the antibody. Unlike the full size IgG4 antibody, the half molecule fragment is very stable and is termed a uniBody. Halving the IgG4 molecule left only one area on the UniBody that can bind to a target. Methods of producing unibodies are described in detail in PCT Publication WO 2007/059782, which is incorporated herein by reference in its entirety (see, also, Kolfschoten et al. (2007) Science 317: 1554-1557).

Affibody molecules are class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (see, e.g., Nord et al. (1997) Nat. Biotechnol. 15: 772-777; Ronmark et al. (2002) Eur. J. Biochem., 269: 2647-2655.). Details of Affibodies and methods of production are known to those of skill (see, e.g., U.S. Pat. No. 5,831,012 which is incorporated herein by reference in its entirety).

It will be recognized that the antibodies described above can be provided as whole intact antibodies (e.g., IgG), antibody fragments, or single chain antibodies, using methods well known to those of skill in the art. In addition, for in vivo applications in human subjects, it is desirable to use a human, humanized, or chimeric human antibody.

Uncrosslinked liposomes with primary antibody/antibody fragments have been documented for therapeutic applications.^{18, 20, 21} One derivative nanoconstruct with quantum dots has been described by one of the inventors with others in Weng, K C et al., “Targeted tumor cell internalization and imaging of multifunctional quantum dot-conjugated immunoliposomes in vitro and in vivo”, Nano Lett. 2008 September; 8(9):2851-7, herein incorporated by reference in its entirety for all purposes. Also of interest is WO 2010/040062, herein incorporated by reference in its entirety for all purposes.

Various reagents and methods known in the art for antibody fragmentation find use to generate moieties that are capable of recognizing and binding specific antigens and/or presenting specific functional groups for site-directed conjugation. In one embodiment, free sulfhydryl groups are generated through controlled reduction. Most bioconjugation involving antibodies uses primary amine groups on antibodies that are not “site-specific”, i.e., the location and number of primary amines are undetermined. Second, not all antibodies are the same, in fact, they are very different in terms of their chemical and physical properties and how amenable they are to modification by certain chemicals/reagents. Therefore, conditions used for one antibody may not be applicable to another antibody; the conditions used for different primary and secondary antibodies fragmentation are indeed very different. An additional consideration is, fragmentation of primary antibody for therapeutic applications, the goal is to remove Fc region and obtain Fab′ fragments with free sulfhydryls. In one embodiment, one does not concern whether Fc regions is intact and attached to the primary or secondary antibody fragments because immunodetection should not be affected by the presence of Fc.

### 4. Detectable Label or Reporting Moiety

In various embodiments, the present target nanocluster or nanoaggregate compositions further comprise a detectable label or reporting moiety component. Illustrative detectable labels include without limitation a fluorescent label, an enzyme, a colorimetric label, a luminescent label, a radioactive label, a radiopaque label or a contrast agent, an MRI label, an electron spin label, and a magnetic label.

### Photoluminescent Labels

In various embodiments, the detectable label can be a photoluminescent component. The photoluminescent component can be any known or available probe, metal, semiconductor material, radioactive label, enzyme, protein or biomolecule which can be detected by photodetection.

An illustrative photoluminescent component is a nanocrystal of semiconducting materials, including without limitation “quantum dots” (QDs), quantum rods (QRs), and quantum wires (QWs). QDs, QRs, and QWs have several advantages over conventional fluorescent dyes, including a long luminescent lifetime and near quantitative light emission at a variety of preselected wavelengths. QDs typically contain a semiconductor core of a metal sulfide or a metal selenide, such as zinc sulfide (ZnS), lead sulfide (PbS), or, most often, cadmium selenide (CdSe). Non-heavy metal-based QDs have also been reported. Upconverting QDs have also been reported.²² The semiconductor core may be capped with tiopronin or other groups or otherwise varied to modify the properties of the quantum dots, most notably to vary biocompatibility and enhance chemical versatility. The emission wavelengths of nanoparticles may be between about 400 nm and about 900 nm, including but not limited to the visible range, and the excitation wavelength between about 250 nm and 750 nm.

QDs typically have diameters of 1 to about 20 nm, depending on the emission wavelength desired, thickness of coating, and the particular application for the targeted nanoclusters or nanoaggregates. In freeze-fracture electron microscopy characterization, the shadow cast by QDs is evidence of their hard-core structure.¹⁶ One or more QDs can be conjugated to a single nanoscaffold. The number of QDs attached to a nanoscaffold may be, e.g., at least 2, 3, 4, 5, 10, 25, 50, 100, 200, 300, 400, 500, 600, 700, 800, 900, 1000 or more, as allowed by the surface area of the nanoparticle core particle, steric effects of adjacent QDs, and the number of functional groups present on the nanoscaffolds. The QDs on a particular nanocluster or nanoaggregate may be of a single color (i.e., single predominant emission wavelength), or of a plurality of colors.

A selected set of QDs may be attached to a nanoscaffold in a multiplexed manner to produce nanoparticle labeling reagents with a “bar code,” i.e., an emission spectra characterized by particular emission wavelengths and intensities (both relative and absolute). Such labeling reagents can be resolved by spectral unmixing techniques and used for, e.g., (i) multi-color labeling, (ii) multi-color coding, (iii) multiple parameter diagnosis, and the like.

Commercially available (off-shelf) QDs include peak emission at 525 nm, 545 nm, 565 nm, 585 nm, 605 nm, 625 nm, 655 nm, 705 nm, and 800 nm.

In one embodiment, the inorganic core comprises a fluorescent semiconductor nanocrystal or metal nanoparticle. The term “nanoparticle” as used herein refers to a particle whose size is measured in nanometers. Nanoparticles include without limitation, e.g., semiconductor nanocrystals, metal nanocrystals, hollow nanoparticles, carbon nanospheres. The nanoparticles can be of any shape including, rods, wire, arrows, teardrops and tetrapods (see, e.g., Alivisatos et al., J. Am. Chem. Soc. 122:12700-12706 (2000)). Other suitable shapes include, e.g., square, round, elliptical, triangular, rectangular, rhombal and toroidal. The nanoparticles typically comprise a shell and a core. Typically the shell material will have a bandgap energy that is greater than the bandgap energy of the core material. In some embodiments, the shell material has an atomic spacing close to that of the core material. The term “monolayer” refers to each atomic layer of the shell material around the core. Each monolayer increases the diameter of the shell material, and increases the emission and total fluorescence of the core. The shell may further comprise a hydrophilic material (e.g., any compound with an affinity for aqueous materials such as H₂O). Nanoparticles include, e.g., semiconductor nanocrystals.

The nanoparticle portion of the conjugates described herein typically comprise a core and a shell. The core and the shell may comprise the same material or different materials. The shell may further comprise a hydrophilic coating or another group that facilitates conjugation of a chemical or biological agent or moiety to a nanoparticle (i.e., via a linking agent). In some embodiments, the semiconductor nanocrystals comprise a core upon which a hydrophilic coating has been deposited.

The core and the shell may comprise, e.g., an inorganic semiconductive material, a mixture or solid solution of inorganic semiconductive materials, or an organic semiconductive material. Suitable materials for the core and/or shell include, but are not limited to semiconductor materials, carbon, metals, and metal oxides. In a preferred embodiment, the nanoparticles comprise a semiconductor nanocrystal. In a particularly preferred embodiment, the semiconductor nanocrystals comprise a CdSe or CdSeTe or InGaP core, and a ZnS shell which further comprises a hydrophilic coating.

The core typically has a diameter of about 1, 2, 3, 4, 5, 6, 7, or 8 nm. The shell typically has thickness of about 1, 2, 3, 4, 5, 6, 7, or 8 nm and a diameter of about 1 to about 10, 2 to about 9, or about 3 to about 8 nm. In a preferred embodiment, the core is about 2 to about 3 nm in diameter and the shell is about 1 to about 2 nm in thickness.

Suitable semiconductor materials for the core and/or shell include, but are not limited to, elements of Groups II-VI (ZnS, ZnSe, ZnTe, CdS, CdSe, CdTe, HgS, HgSe, HgTe, MgS, MgSe, MgTe, CaS, CaSe, CaTe, SrS, SrSe, SrTe, BaS, BaSe, BaTe, and the like) and III-V (GaN, GaP, GaAs, GaSb, InN, InP, InAs, InSb, and the like) and IV (Ge, Si, and the like), and alloys or mixtures thereof. Suitable metals and metal oxides for the core and/or shell include, but are not limited to, Au, Ag, Co, Ni, Fe₂O₃, TiO₂, and the like. Suitable carbon nanoparticles include, but are not limited to, carbon nanospheres, carbon nano-onions, and fullerene. In one embodiment, Au nanoparticles are provided as the core particle.

Semiconductor nanocrystals can be made using any method known in the art. For example, methods for synthesizing semiconductor nanocrystals comprising Group III-V semiconductors or Group II-VI semiconductors are set forth in, e.g., U.S. Pat. Nos. 5,751,018; 5,505,928; and 5,262,357. The size of the semiconductor nanocrystals can be controlled during formation using crystal growth terminators U.S. Pat. Nos. 5,751,018; 5,505,928; and 5,262,357. Methods for making semiconductor nanocrystals are also set forth in Gerion et al., J. Phys. Chem. 105(37):8861-8871 (2001) and Peng et al., J. Amer. Chem. Soc., 119(30):7019-7029 (1997).

The semiconductor nanocrystals may further comprise a hydrophilic coating (e.g., a coating of hydrophilic materials or stabilizing groups) to enhance the solubility of the nanocrystals in an aqueous solution. Typically the hydrophilic coating is about 0.1, 0.25, 0.5, 0.75, 1, 2, 3, 4, 5, 6, 7, 8, 9, or 10 nm thick. Suitable hydrophilic materials include, e.g., SiO, SiO₂, polyethylene glycol, ether, mecapto acid and hydrocarbonic acid, and dihydroxylipoic acid (DHLA). Suitable stabilizing groups include, e.g. positively or negatively charged groups or groups that facilitate steric repulsion. In a preferred embodiment, the hydrophilic coating is a silica shell (e.g., comprising SiO₂). Methods of silanizing semiconductor nanocrystals are well known in the art and are described in, e.g., Gerion et al., Chemistry of Materials, 14:2113-2119 (2002). Other methods for generating water-soluble semiconductor nanocrystals are described in, e.g., Mattoussi et al., Physica Status Solidi B, 224(1):277-283 (2001) and Chan et al., Science, 281:2016-2018 (1998).

In a preferred embodiment, the hydrophilic coating comprises a silica shell having a thickness of about 0.5 to about 5, about 1 to about 4, or about 2 to about 3 nm. Preferably the silica shell is amorphous and porous. Silica shells can be deposited on the core or the shell of the semiconductor nanocrystal using the methods described in, e.g., Alivisatos et al., Science, 281:2013-2016 (1998) and Gerion, et al., J. Phys. Chem. 105(37):8861-8871 (2001). In a preferred embodiment, the semiconductor nanocrystals have core/shell configuration of CdSe/ZnS/SiO₂ wherein the layers are about 25/5/50 Å respectively from the center of the core.

Quantum dot labels that find particular use are those that are biocompatible. Such quantum dots usually have layers of organic coating to make them hydrophilic, biocompatible, and present specific chemical groups. Biocompatible fluorescent nanocrystals refer to core/shell structure quantum dots including CdSe/ZnS, generally having a hydrophilic polymer coating, silica, derivatized surface with biomolecules such as streptavidin, nucleotides, peptides, or chemical groups. See, e.g., Gerion, et al., Journal of Physical Chemistry (2001) 105(37):8861-8871; Pathak, et al., J. Am. Chem. Soc. (2001) 123(17):4103-4104; Chan, et al., Current Opinion in Biotechnology (2002) 13(1):40-46; Larson, et al., Science (2003) 300(5624):1434-1436; Jaiswal, et al., Nature Methods (2004) 1(1):73-78; Jaiswal, et al., Trends in Cell Biology (2004) 14(9):497-504; Alivisatos, et al., Annual Review of Biomedical Engineering (2005) 7:55-76; Bentzen, et al., Bioconjugate Chemistry (2005) 16(6):1488-1494; Parak, et al., Nanotechnology (2005) 16:R9-R25; Selvan, et al., Advanced Materials (2005) 17(13):1620-1625; Jiang, et al., Chemistry of Materials (2006) 18(20):4845-4854. Moreover, Michalet, et al., Science (2005) 307(5709):538-544; and Klostranec, et al., Advanced Materials (2006) 18(15):1953-1964 provide extensive reviews on surface polymer coatings for quantum dots. The surface layer, or multiple layers, of coating provide shielding from the inorganic/semiconductor materials and may also serve desired functionality. The composition and properties are well documented. Reactive functional groups include primary amines, carboxylic acids, alcohols, and thiols.

### Lipophilic Dyes

In another embodiment, lipophilic dyes that non-covalently intercalate in lipid bilayers can also be used for fluorescence probes in targeted nanoclusters or nanoaggregates, although lacking the stability of quantum dots. Commonly used lipidic dyes include 3,3′-dioctadecyloxacarbocyanine perchlorate (‘DiO’; DiOC18(3)), 4-(4-(dihexadecylamino)styryl)-N-methylpyridinium iodide (DiA; 4-Di-16-ASP), 1,1′-dioctadecyl-3,3,3′,3′-tetramethylindocarbocyanine perchlorate (‘DiI’; DiIC18(3)), 1,1′-dioctadecyl-3,3,3′,3′-tetramethylindodicarbocyanine perchlorate (‘DiD’ oil; DiIC18(5) oil), 1,1′-dioctadecyl-3,3,3′,3′-tetramethylindotricarbocyanine iodide (‘DiR’; DiIC18(7)), etc. Molecular Probes, Invitrogen (Carlsbad, Calif.) is one of the major commercial sources for such lipophilic tracers.

### Other Detectable Labels

Other detectable labels are also suitable for use in this invention. Detectable labels suitable for use include any composition detectable by spectroscopic, photochemical, biochemical, immunochemical, electrical, optical or chemical means. Useful labels in the present invention include labeled beads (e.g., Luminex beads), magnetic beads (e.g., Dynabeads™), fluorescent dyes (e.g., fluorescein isothiocyanate, texas red, rhodamine, green fluorescent protein, and the like), radiolabels (e.g., ³H, ¹²⁵I, ³⁵S, ¹⁴C, or ³²P), radiopaque labels, enzymes (e.g., horse radish peroxidase, alkaline phosphatase and others commonly used in an ELISA), magnetic resonance imaging (MRI) labels, Positron Emission Tomography (PET) labels, and colorimetric labels including colloidal gold or colored glass or plastic (e.g., polystyrene, polypropylene, latex, etc.) beads.

Means of detecting such labels are well known to those of skill in the art. Thus, for example, radiolabels may be detected using photographic film, scintillation detectors, and the like. Fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

### Radioisotope Labels

In various embodiments, the detectable label is a radioisotope. Radiolabels that find use include without limitation ³H, ¹²⁵I, ³⁵S, ¹⁴C, ³²P, ⁹⁹Tc, ²⁰³Pb, ⁶⁷Ga, ⁶⁸Ga, ⁷²As, ¹¹¹In, ^{113m}In, ⁹⁷Ru, ⁶²Cu, ⁶⁴Cu, ⁵²Fe, ^{52m}Mn, ⁵¹Cr, ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, and ¹¹¹Ag.

In certain specific embodiments, this invention contemplates the use of targeted nanoclusters or nanoaggregates for the detection of tumors and/or other cancer cells. Thus, for example, the targeted nanoscaffolds of this invention can be conjugated to gamma-emitting radioisotopes (e.g., Na-22, Cr-51, Co-60, Tc-99, I-125, I-131, Cs-137, Ga-67, Mo-99) for detection with a gamma camera, to positron emitting isotopes (e.g., C-11, N-13, O-15, F-18, and the like) for detection on a Positron Emission Tomography (PET) instrument, and to metal contrast agents (e.g., Gd containing reagents, Eu containing reagents, and the like) for magnetic resonance imaging (MRI).

In various embodiments, the radioisotope labels are attached to the nanoscaffold via a chelating agent. Illustrative chelating agents include those from the polyamino carboxylic acid family of ligands, e.g., DTPA (Pentetic acid or Diethylene triamine pentaacetic acid) and EDTA (Ethylenediaminetetraacetic acid). Another chelating agent that finds use is DOTA (1,4,7,10-tetraazacyclododecane-1,4,7,10-tetraacetic acid).

### Imaging Agents

The nanoscaffolds described herein, can be attached to one or more imaging agents. In various embodiments the imaging agent can be an MRI imaging agent, a PET imaging agent, a NIR imaging agent, and ESR imaging agent, and the like.

### Magnetic Resonance Imaging (MRI) Imaging Agents

In certain embodiments, the imaging agent(s) comprise an MRI imaging agent attached to the nanoparticle.

The MRI imaging agents can include, but are not limited to positive contrast agents and/or negative contrast agents. Positive contrast agents cause a reduction in the T₁ relaxation time (increased signal intensity on T₁ weighted images). They (appearing bright on MRI) are typically small molecular weight compounds containing as their active element Gadolinium, Manganese, or Iron. All of these elements have unpaired electron spins in their outer shells and long relaxivities. A special group of negative contrast agents (appearing dark on MRI) include perfluorocarbons (perfluorochemicals), because their presence excludes the hydrogen atoms responsible for the signal in MR imaging.

In certain preferred embodiments, the MRI imaging or detection agent attached to the present nanoscaffolds are iron or paramagnetic radiotracers and/or complexes, including but not limited to gadolinium, xenon, iron oxide, copper, Gd³⁺-DOTA (DOTA=1,4,7,10-tetrakis(carboxymethyl)-1,4,7,10-tetraazacyclododecane), and ⁶⁴Cu2+-DOTA.

### Positron Emission Tomagraphy (PET) Imaging Agents

The targeted nanoclusters or nanoaggregates also find use in single photon emission computer tomography (SPECT), near infrared (NIR), electron spin resonance (ESR) imaging, and positron emission tomography (PET) imaging. A number of PET imaging radionuclides are known to those of skill in the art. These include, but are not limited to PET radiopharmaceuticals such as [¹¹C]choline, [¹⁸F]fluorodeoxyglucose (FDG), [¹¹C]methionine, [¹¹C]acetate, and [¹⁸F]fluorocholine as well as other radionuclides including but not limited to ¹¹C, ¹⁵O, ¹³N, ¹⁸F, ³⁵Cl, ⁷⁵Br, ⁸²Rb, ¹²⁴I, ⁶⁴Cu, ²²⁵Ac, ¹⁷⁷Lu, ¹¹¹In, ⁶⁶Ga, ⁶⁷Ga, ⁶⁸Ga, and the like.

### Nuclear Magnetic Resonance (NMR) and Electron Spin Resonance Imaging Agents.

In certain embodiments the imaging agents comprise nuclear magnetic resonance (NMR) and/or electron spin resonance imaging agents. Such agents are well known to those of skill in the art and include, for example, nitroxides, and the like. In certain embodiments single-crystal ferrimagnetic spheres offer the advantages of high detectability through large magnetizations and narrow FMR lines. For example, yttrium-iron garnet Y₃Fe₅O₁₂ and γ-Fe₂O₃ are two well-known materials suitable for this application. Different dopants can be added to lower the spin resonance frequencies of these materials for medical applications. Magnetic garnets and spinels are also chemically inert and indestructible under normal environmental conditions. These examples are intended to be illustrative and not limiting.

### Optical Near Infra-Red (NIR)-Based Tissue Imaging

For in vivo optical imaging, the major challenge is that the dyes need to compete for light against the autofluorescing and light scattering nature of tissue, and the strong absorption profiles of biomolecules that absorb mostly in the visible region of the spectrum. The poor penetration of light through tissue limits the uses of these tags to subsurface locations, or requires specialized instrumentation such as a light probe. Theoretical calculations have proposed that NIR excitation light can penetrate tissue between 7-14 cm in depth with sensitive photon collection systems. In view of these observations, fluorophores have been developed that absorb in the NIR of the spectrum (650-900 nm).

Illustrative NIR dyes include a cyanine or indocyanine derivative. Such dyes include, but are not limited to Cy5.5, IRDye800, indocyanine green (ICG), indocyanine green derivatives and combinations thereof. In one specific embodiment, the dye includes a tetrasulfonic acid substituted indocyanine green (TS-ICG) (see, e.g., U.S. Pat. No. 6,913,743). Examples of suitable indocyanine include ICG and its derivatives. Such derivatives can include TS-ICG, TS-ICG carboxylic acid and TS-ICG dicarboxylic acid.

Additional examples include dyes available from Li-Cor, such as IR Dye 800CW™, available from Li-Cor. Additional examples include dyes disclosed in U.S. Pat. No. 6,027,709. In one embodiment, the dye is N-(6-hydroxyhexyl)N′-(4-sulfonatobutyl)-3,3,3′,3′-tetramethylbenz(e)indo-dicarbocyanine, and/or N-(5-carboxypentyl)N′-(4-sulfonatobutyl)3,3,3′,3′-tetramethylbenz(e)indod-icarbocyanine

These dyes have a maximum light absorption which occurs near 680 nm. They thus can be excited efficiently by commercially available laser diodes that are compact, reliable and inexpensive and emit light at this wavelength. Suitable commercially available lasers include, for example, Toshiba TOLD9225, TOLD9140 and TOLD9150, Phillips CQL806D, Blue Sky Research PS 015-00 and NEC NDL 3230SU. This near infrared/far red wavelength also is advantageous in that the background fluorescence in this region normally is low in biological systems and high sensitivity can be achieved.

In certain embodiments the nanoscaffolds may be conjugated to a lissamine dye, such as lissamine rhodamine B sulfonyl chloride. Lissamine dyes are typically inexpensive dyes with attractive spectral properties. For example, examples have a molar extinction coefficient of 88,000 cm⁻¹M⁻¹ and good quantum efficient of about 95%. It absorbs at about 568 nm and emits at about 583 nm (in methanol) with a decent stokes shift and thus bright fluorescence.

In one embodiment, the detection and NI imaging agent used in the multimodal probe is NIRQ820, a cyclohepta polymethine fluorochrome, ex/em=790/820 nm, a water soluble NIR fluorochrome with great chemical stability.

### Radiopaque Labels or Contrast Agents

In certain embodiments, the detectable label is a “radiopaque” label, e.g., a label that can be easily visualized using x-rays. Radiopaque materials are well known to those of skill in the art. The most common radiopaque materials include iodide, bromide or barium salts. Other radiopaque materials are also known and include, without limitation organic bismuth derivatives (see, e.g., U.S. Pat. No. 5,939,045), radiopaque polyurethanes (see U.S. Pat. No. 5,346,9810, organobismuth composites (see, e.g., U.S. Pat. No. 5,256,334), radio-opaque barium polymer complexes (see, e.g., U.S. Pat. No. 4,866,132), and the like.

### 5. Formation of Targeted Nanoclusters or Nanoaggregates

The nanoclusters or nanoaggregates described herein are comprised of aggregated or crosslinked polyvalent nanoparticle core units or nanoscaffolds. The present invention is based, in part, on the achievement of signal amplification through crosslinked nanoparticle core units. Crosslinking allows the focused delivery of more functional components, e.g., targeting moieties and detectable moities, than possible using prior technologies. The nanoclusters or nanoaggregates are crosslinked to an extent sufficient to accomplish stable association of multiple core units; to achieve amplified delivery of functional components without compromising the function of the components; and to form a composition that is a uniform suspension, i.e., a composition with no or without substantial phase separation or nanocluster or nanoaggregate precipitation. The nanoclusters or nanoaggregates are of a size and level of crosslinking to avoid increased bulkiness that tends to create steric hindrance for the nanocluster or nanoaggregate to access its targets and inhomogeneous suspension in physiological buffers, e.g., of pH in the range of about pH 5-9.

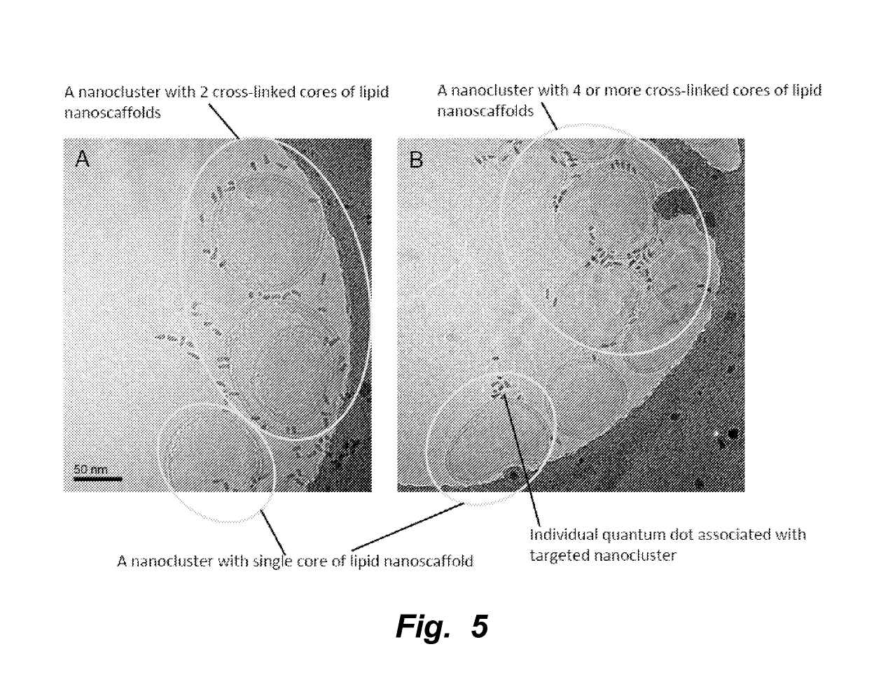

In various embodiments, the nanoclusters or nanoaggregates can be in the size range of 10 nm to 10 μm, and can include in the range of about 2 to about 200, or more, polyvalent nanoparticle core units or nanoscaffolds. For example, a composition comprising a population of nanoclusters or nanoaggregates may comprise nanoclusters or nanoaggregates having an average or median number of about 2, 3, 4, 5, 10, 20, 25, 50, 75, 100, 125, 150, 175, 200 or more polyvalent nanoparticle core units or nanoscaffolds. Surface area for attachment of increased numbers of functional groups can be achieved by crosslinking or aggregating smaller polyvalent nanoparticle core structures or nanoscaffolds. In a preferred embodiment, polyvalent nanoparticle core structures nanoscaffolds with an average diameter of less than about 100 nm, for example, less than about 90 nm, 80 nm, 70 nm, 60 nm, 50 nm, 40 nm, 30 nm, 20 nm, are cross-linked or aggregated into a nanocluster. Each polyvalent nanoparticle core structure or nanoscaffold can be attached to a number of targeting moieties in the range of about 1 to about 100,000 (for example, about 2, 5, 10, 25, 50, 100, 200, 500, 1000, 5000, 10,000, 50,000, 100,000 targeting moieties) and detectable labels in the range of about 1 to about 100,000 (for example, about 2, 5, 10, 25, 50, 100, 200, 500, 1000, 5000, 10,000, 50,000, 100,000 detectable labels). In some embodiments, the nanoparticle core structures are attached to on average more than 10, e.g., more than 20, detectable labels and more than 500 targeting moieties. Illustrative crosslinked or aggregated nanoclusters or nanoaggregates are shown by cryo-electron microscopy images in FIG. 5.

The nanoclusters or nanoaggregates can be prepared by providing nanoscaffold core units with multiple and distinct functional groups on the outer surface for cross-linking to the target moieties and to the detectable labels. That is, a nanoscaffold core unit with a first functional group for conjugating to a targeting moiety and a second functional group for conjugating to a detectable label is provided, wherein the first and second functional groups are different. In various embodiments, a third functional group optionally can be incorporated into the nanoscaffolds, for crosslinking between two or more nanoscaffolds. Illustrative functional groups included without limitation carboxyl, alcohol, amine, amino, thiol, disulfide, urea, or thiourea groups, which then allow chemical linkage of the nanoscaffold core units and functional components (i.e., targeting moieties and detectable labels).

Methods for derivatizing nanoscaffolds, including liposomes, dendrimers, metal particles, and other particles are known in the art and can be found, e.g., in Rhyner, et al (2006) Nanomedicine 1:209-17; Jamieson (2007) Biomaterials 28:4717-32; Iga (2007) J Biomed Biotech 2007:76087-97; Zhou, et al (2007) Bioconjugate Chemistry 18:323-32; Tortiglione, et al (2007) Bioconjugate Chemistry 18:829-35, Setvan, et al (2007) Angewandte Chemie International Edition 48:2448-52; Kampani, et al (2007) J Virological Methods 141:125-32; Medintz, et al (2007) Nano Letters 7:1741-48; de Farias, et al (2005) J Microscopy 219:103-08; Gao, et al, (2002) J Biomedical Optics 7:532-37, Tan, et al (2007) Biomaterials 28:1565-71; Allen, et al (1995) Biochim Biophys Acta 1237:99-108; and Hansen, et al (1995) Biochim Biophys Acta 1239:133-44, and in U.S. Pat. Nos. 7,138,121; 7,133,725; 7,112,337; 7,108,883; 6,369,206; 5,861,319; 5,714,166 and 5,468,606. A method for forming nanoparticle labeling reagents using 1-ethyl-3-(3-dimethylaminopropyl)carbodiimide is described by Sheehan, et al. (1957) J Am Chem Soc 79:4528-429.

To provide a non-limiting example, maleimide and amino functional groups can be incorporated into lipid vesicles. The maleimide groups on the nanoscaffold can be conjugated to sulfhydryl groups on reduced antibodies and the amino groups on the nanoscaffold can be conjugated to carboxyl groups on quantum dots. The nanoclusters or nanoaggregates are crosslinked to maximize stable association of multiple nanoparticle core units and the number of attached detectable labels, while minimizing non-uniform populations of nanoclusters or nanoaggregates and steric hindrance of nanoclusters or nanoaggregates when applied to detection.

The choice of crosslinkers depends on the available functional groups on the surface of the functional components (e.g., the targeting moieties and the detectable labels) and the nanoscaffold core units. Crosslinkers are selected that properly activate the functional groups on components, and do not interfere with or neutralize the conjugations of multiple functional components onto the nanoscaffold or the crosslinking between nanoscaffolds. To return to the non-limiting example of a nanoscaffold core unit crosslinked to an antibody targeting moiety and a quantum dot detectable label, EDC/Sulfo-NHS was used to link the amine groups on the nanoscaffold and the carboxyl groups on the quantum dots; maleimide groups on the nanoscaffold were linked to sulfhydryl groups on reduced antibodies. The crosslinking reaction of either the targeting moiety or the detectable label to the nanoscaffold can also serve to crosslink two or more nanoscaffolds into a nanocluster. To return to the non-limiting example of a nanoscaffold core unit crosslinked to an antibody targeting moiety and a quantum dot detectable label, EDC/Sulfo-NHS crosslinked the quantum dot to the nanoscaffold and also crosslinked two or more nanoscaffolds into a nanocluster.

Sequential conjugation of targeting moieties and detectable labels can be used to stably link the functional components to nanoscaffolds. The conjugation reactions of the targeting moieties and the detectable labels should not interfere with each other or interfere with the function of individual components.

The size and extent of crosslinking can be controlled in several stages during synthesis and the conditions used in the conjugation reactions. First, the size of the nanoparticle core is controllable. In the case of lipid vesicles, the synthesis procedure has been well documented, and is described herein. In the process of forming lipid vesicles, sonication and/or extrusion through various sizes of electron-etched polycarbonate membranes can achieve narrow distribution of the resulting lipid vesicles. Second, the conditions for crosslinking can be controlled during the conjugation between nanoscaffolds and functional components. Adjustable conditions can include the concentration of cross-linker, the time of exposure of the nanoscaffolds and functional components to the crosslinker, the stoichiometry of the nanoscaffold and the functional component, and the pH of the reaction solution. To continue with the non-limiting example of a nanoscaffold core unit crosslinked to an antibody targeting moiety and a quantum dot detectable label, (a) the concentration of EDC and Sulfo-NHS can be adjusted to yield various size and size distribution of nanoclusters or nanoaggregates if the functional components are linked via amine and carboxyl groups; (b) the ratio (stoichiometry) of nanoparticle scaffold and functional components can be adjusted to achieve desired association of various chemical entities involved; and (c) the pH of media/buffer used for the reactions and incubation time. To minimize population broadening, a lower transient concentration of crosslinkers can be used, rather than a higher overall concentration. One set of conditions for crosslinking amine and carboxyl groups is to maintain a transient concentration of about 0.1-1.0 mM of EDC and sulfo-NHS, and the total amount of EDC/Sulfo-NHS achieves an overall concentration of about 2-10 mM at the end of the conjugation reactions.

In one embodiment, a targeted nanocluster or nanoaggregate is formed by the protocol shown below. Briefly, the general steps for construction include:
(a) providing the prepared lipid nanoscaffold;
(b) providing the reduced or derivitized targeting moiety (e.g., antibody or antibody fragment);
(c) attaching the targeting moiety to the lipid nanoscaffolds; and
(d) conjugating the antibody-nanoscaffolds to the detectable label.

The order of steps (c.) and (d.) can be interchanged, i.e., detectable labels can be conjugated to the nanoscaffolds, followed by linking with the targeting moiety. Crosslinking between the nanoscaffolds can be achieved concurrently with the crosslinking of either the targeting moiety or the detectable label. In such cases, the crosslinker that also can be used in either step (c.) or step (d.). Alternatively, crosslinking of two or more nanoscaffolds to form a nanocluster or nanoaggregate can be performed in a separate crosslinking step subsequent to crosslinking the targeting moieties and the detectable labels to the nanoscaffolds. In some embodiments, detectable labels can be incorporated into nanoscaffolds by means other than conjugation or cross-linking, by e.g., encapsulation, embedding, active loading, passive loading, crystallization, electrostatic interactions, or binding pair interactions (e.g., avidin-biotin conjugation), before installation of targeting moieties and crosslinking of nanoscaffolds).

The following is a sample protocol for the formation of a targeted nanocluster or nanoaggregate comprising a lipid vesicle nanoscaffold attached to a secondary antibody targeting moiety and a quantum dot detectable label:

(1.) Lipid vesicle nanoscaffolds preparation
a. Buffer: 5 mM HEPES or 5 mM phosphate, 135 mM NaCl, pH 7
b. Exemplary concentration: 30 mg/ml
c. Add ˜11 ml chloroform and ˜1 ml methanol
d. Vortex to mix
e. Use a rotary evaporator to remove solvent
f. Vacuum for ˜45 min
g. Freeze-dry ˜460 mTorr ˜56° C. for 1 hr 30 min
h. Add 10 ml of buffer to the dried lipid film
i. Thaw at 60° C. and freeze on dry ice. Freeze-thaw multiple times
j. Extrude through a 100 nm PC membrane at ˜60° C. 11 times by extruder
k. Store at 4° C.

(2.) Reduction of targeting moiety, e.g., secondary antibody
a. Prepare 50 mM DTT, 2-ME, or 2-MEA solution.
b. Exemplary antibody: Goat anti-mouse IgG (GAM)
c. Reduce GAM by adding 100 μA 50 mM 2-ME, 2-MEA, or DTT to 400 μA GAM and incubate at 37° C. for 30 min
d. Transfer to ice water bath and desalt by spin columns
e. Store at 4° C.

(3.) Preparation of goat anti-mouse nanocluster
a. Add ˜400 μA reduced GAM to ˜575 μA lipid vesicles
b. Vortex
c. Incubate at RT for several hours
d. Store at 4° C.
e. Purify by dialysis or chromatography

(4.) Preparation of QD conjugated goat anti-mouse nanocluster
a. Add 50 μA 8.0 μM carboxyl Qdots to 500 μA of goat anti-mouse secondary immunoliposomes
b. Prepare 50 mM 1-ethyl-3-(3-dimethylaminopropyl)carbodiimide hydrochloride (EDC), and sulfo-NHS
c. Add an overall 40 μl of 50 mM EDC and 40 μA 100 mM sulfo-NHS
d. Place on a rocking shaker at RT for 4-12 hr
e. Store at 4° C.

In another embodiment, purification for targeted nanoclusters or nanoaggregates can be achieved by size-exclusion gel chromatography or dialysis through polycarbonate membranes with suitable pore sizes and cut-off molecular weights.

Even if not expressly enumerated, the present invention contemplates nanoclusters or nanoaggregates composed of all possible combinations of the listed nanoscaffolds, targeting moieties and detectable moieties discussed herein. Accordingly, any of the various nanoscaffolds can be combined with any of the various targeting moieties and any of the various detectable moieties, and optionally with a therapeutic agent.

### 6. Applications for Targeted Nanoclusters or Nanoaggregates

In one embodiment, the targeted nanoclusters or nanoaggregates can be used for diagnostic and immunodetection purposes. One aim in a targeted nanocluster or nanoaggregate for diagnostics or detection is to achieve incorporating as many detectable reporters in a single nanocluster or nanoaggregate as possible without compromising the structural integrity and the targeting capability of the nanoparticle. The targeted nanoclusters or nanoaggregates can generally be used in place of the primary and/or secondary antibody in immunoassays, including without limitation flow cytometry, enzyme-linked immunosorbent assay (ELISA), Western blot assays, dot blot assays, immunohistochemistry, immunocytochemistry, mass cytometry, capillary electrophoresis, microbead assays.

Targeted nanoclusters or nanoaggregates offer higher sensitivity with signal amplification; therefore, for rare and hard-to-detect antigens and/or in unconventional detection schemes, targeted nanoclusters or nanoaggregates offer unique advantages. One non-limiting example is high-resolution capillary isoelectric focusing.²³ Due to the extremely small amounts of samples being detected, fluorescence was not adopted in prior applications; instead, chemiluminescence was chosen for reporting. With signal amplifying properties, targeted nanoclusters or nanoaggregates improve the method for capillary isoelectric focusing. Targets of interest include those described above and additionally, e.g., FOXO3a, SIRT, GITR, AKT, pAKT, pmTOR, Bcl-6, CD10, EGFR, HER2, HER3, CK5/6, CK17, Estrogen receptor (ER) and Progesterone receptor (PR). Such targets can be detected using the nanoclusters in any applicable detection assay known in the art, including immunohistochemistry assays. For example, diagnostic immunostaining of human epidermal growth factor receptor 2 (HER2/erbB2) on cell and tissue sections by targeted nanoclusters or nanoaggregates with a simplified procedure is demonstrated herein. Targeted nanoclusters or nanoaggregates achieved specific, efficient and quantitative immunostaining by showing fluorescence images and intensities corresponding to known HER2 expression levels, i.e., quantities of HER2 receptors per cell.

Secondary targeted nanoclusters or nanoaggregates, described herein, can be derived from secondary antibody against primary antibodies, thus eliminating the need for optimization for labeling for different antigens/markers and potentially allowing a higher working dilution for primary antibody.⁵ The primary antibody can often be used at a higher working dilution in the indirect binding method than the direct method to achieve successful staining. This is an inherent advantage associated with secondary amplification and also applies to some traditional methods such as avidin-biotin complex ‘ABC’, peroxidase anti-peroxidase ‘PAP’, or polymer-based reagents mentioned above.

The fragmentation of antibody fragments used in the targeted nanoclusters or nanoaggregates can be achieved by various fragmentation techniques known in the art. Immunoglobulin IgG consists of multiple components that can be digested and reduced. Various chemical agents and conditions exist for this purpose, yet results vary widely for different antibodies.

To achieve sensitive and quantitative detection of tumor biomarkers through localization of luminescent quantum dots (QDs), it is desirable that targeted nanoclusters or nanoaggregates specifically and efficiently bind to the target antigens. Several advantages are achieved by the presently described targeted nanoclusters or nanoaggregates.

The fluorescence images and intensities should reflect the localization expression levels of the antigens, respectively. By attaching multiple antibody fragments, avidity effect (one antigen binding facilitates the neighboring antigen bindings and multiple antigen binding sites simultaneously interact with a target through combined synergistic strength of bond affinities) takes place which further enhances specific binding of targeted nanoclusters or nanoaggregates.

Also by attaching a multiplicity of QDs, amplification of signals is realized. One advantage of the present targeted nanoclusters or nanoaggregates is the ability to form aggregates which lends greater signal amplification, changes in light scattering properties of the nanoclusters or nanoaggregates, and separation of a target using such known techniques as chromatography or electrophoresis. Furthermore, by employing a fluorescence modality, the dynamic range of detection is improved compared to traditional chromogenic approaches.

The presently described targeted nanoclusters or nanoaggregates find broad applicability in many fields of application which currently rely on immunofluorescence amplification. These include but are not limited to, immunohistochemistry, immunocytochemistry, flow cytometry, microarrays, enzyme-linked immunosorbent assay (ELISA), Western blot assays, dot blot assays, fluorescent in situ hybridization (FISH), bead-based assays (e.g., Luminex Bead Assays, polystyrene beads), high-resolution capillary isoelectric focusing (Firefly system)²³, and any other technique and biological assays involving antibody recognition of antigen, proteins, pathogens, and nucleotides (DNA and RNA).

In another embodiment, the targeted nanoclusters or nanoaggregates can be used for therapeutic purposes. Targeted nanoclusters can be used to deliver therapeutic agents (e.g., an anticancer or antineoplastic agent). The therapeutic agent can be loaded within the nanoscaffolds. The detectable agents can be used to monitor delivery of the therapeutic agent to the target site. In some embodiments, the targeted nanoclusters find use in in vivo imaging methods, e.g., MRI, PET scans, CAT scans, x-ray, and other known imaging techniques, as described herein. For therapeutic and in vivo purposes, the nanoclusters are administered to a subject. The route of administration will depend the intended target for the nanocluster. The route chosen will allow the targeted nanocluster to bind to its intended target. Illustrative routes of administration include, e.g., isophoretic delivery, transdermal delivery, aerosol administration, administration via inhalation, oral administration, intravenous administration, intraperitoneal administration and rectal administration. Dosing will depend on several variables, including, e.g., the intended use (imaging or therapy), the route of administration, the weight of the subject, among others. In determining an effective dose, a low initial dose of nanoclusters can at first be administered. The dose can then be incrementally increased until the desired effect is achieved with minimal or no adverse side effects. As appropriate, the nanoclusters can be administered once or in multiple administrations. To provide an example, nanoclusters comprised of lipidic nanoparticles can be administered intravenously with a corresponding lipid dose of about 0.6-1.5 μmol of phospholipid and 1.8-4.0 μmol total in three injections. Typically, a dose in the range of 10-200 μl is injected into a 20 g mouse. In nanoclusters encapsulating a therapeutic agent, about 5.0-10.0 mg therapeutic agent/kg/dose can be administered every week for 3 weeks, for a total therapeutic agent dose of about 15.0-30.0 mg/kg). This dose can be adjusted to be higher or lower, as appropriate for a particular therapeutic agent.

Uses of the invention include research, pharmaceutical companies (drug discovery and personalized medicine), pathology laboratories, hospitals, and educational facilities. In such settings, specimens treated by targeted nanoclusters or nanoaggregates can be examined by conventional flow cytometry/FACS, fluorescence microscopy, confocal microscopy, spectrofluorometry, and/or any technique that collects and analyzes fluorescence signals. To examine cell and tissue sections treated by targeted quantum dot nanocluster, a fluorescence imager, such as fluorescence microscope or confocal laser scanning microscope is used. The microscopes should provide the appropriate lasers for excitation of the targeted quantum dot nanoclusters or nanoaggregates, and for any staining performed to the specimens in order to visualized specific features of interest. The microscopes should also be installed with appropriate filters or spectral window selectors for filtering out background/excitation lights and allow only the fluorescence of targeted nanocluster or nanoaggregate or any emitters of interest to be collected. In one example, confocal laser-scanning microscopy was performed using Zeiss LSM 710 laser scanning microscope equipped with a diode 405-30 laser (wavelength=405 nm, maximum power=30.0 mW), an argon laser (wavelengths=458, 488, and 514 nm, maximum power=25.0 mW), a DPSS 561-10 laser (wavelength=561 nm, maximum power=15.0 mW), and a HeNe 633 (wavelength=633 nm, maximum power=5.0 mW). Typically, a diode 405 nm laser is used for DAPI nucleus stain excitation. For example, a diode 405 nm or argon 488 nm laser was used for quantum dot nanocluster or nanoaggregate excitation. Formalin-fixed, paraffin-embedded (FFPE) cell and tissue sections on plus glass slides were processed to remove paraffin and subjected to antigen retrieval, blocking, primary antibody binding, and secondary quantum dot nanocluster or nanoaggregate binding, and finally a droplet of Vectashield antifade mounting medium including DAPI (Vector Laboratories, Burlingame, Calif.) was applied immediately afterwards, followed by sandwiching with a cover glass. 20× and 40× magnification objectives were used for inspection and scanning

### EXAMPLES

The following examples are offered to illustrate, but not to limit the claimed invention.

### Example 1

### Sample Prior Art or Conventional Immunostaining

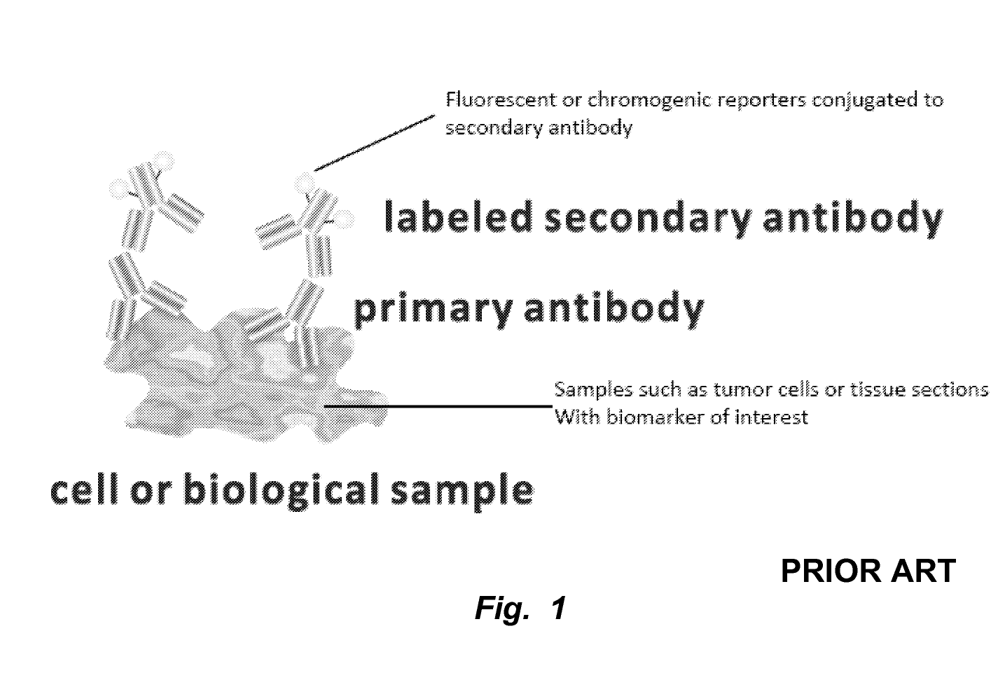

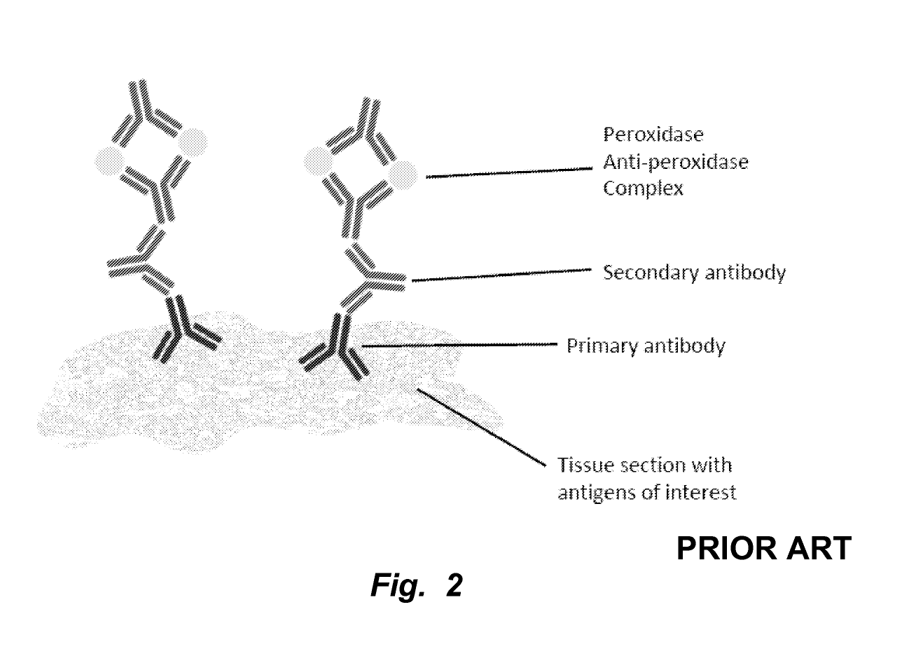

Conventional methods of immunostaining are illustrated in FIGS. 1 and 2. In the method illustrated, tissues, cells, or cell material including an antigen of interest are immobilized on a solid support, such as a glass slide, well of a multi-well plate, or the like and incubated in the presence of a primary antibody specific for the antigen of interest. After removing unbound primary antibody by washing, secondary antibody is allowed to bind to the primary antibody, followed by addition of antibody-color development reagent complexes capable of acting on a substrate to produce a detectable signal that corresponds, indirectly, to the present of the antigen of interest.

The conventional method is time-consuming, with typical processing times being about two days. Several discrete binding steps are required, e.g., for binding of primary antibody, secondary antibody, colorimetric development agents, and counterstaining. Moreover, the results are generally not quantitative.

### Example 2

### Flow Cytometry of Live Breast Tumor Cells Using Commercially Available Qdot IgG Conjugate and Targeted Nanoclusters or Nanoaggregates

Synthesis of targeted nanoclusters or nanoaggregates was achieved by incorporating reduced antibody binding fragments and luminescent quantum dots into a surface-bifunctionalized and cross-linked lipid vesicle scaffold. An illustrative protocol using the present composition and method for labeling live breast tumor cells is described.
1. Take ˜70% confluent flasks of cancer cells.
2. Aspirate the old medium from the flask, wash with PBS and trypsinize the cells with 0.25% trypsin-EDTA. Finally, neutralize trypsin by adding back media.
3. Collect the cell suspension in a 50 ml centrifuge tube. Count the cells with hemocytometer.
4. Aliquote appropriate volume of cell suspension into labeled Eppendorf tubes, typically 150,000 cells/tube.
5. Spin down the cells at 400×g for 5 min, remove the supernatant and add 100 μl of 1% BSA in 1×PBS+10% FBS in each tube on ice.
6. Gently vortex to resuspend cells.
7. Incubate with primary antibody, typically 1-5 μg/ml. For example, Invitrogen Cat. No. 28-0003Z mouse anti-HER2 (c-erbB-2), Clone TAB250: the starting concentration is 75 μg/ml. Add 1.5 μl anti-HER2MAb to each Eppendorf tube with 100 μl 1% BSA in 1×PBS in it.
8. Mix well and incubate on ice for 1 hr. Gently vortex 1-2 times during the 1 hr incubation.
9. Centrifuge at 400×g for 5 min and remove suspension and add fresh 1% BSA in 1×PBS. Repeat once.
10. Add 40 μl pre-diluted Qdot IgG conjugate or targeted Qdot nanoclusters or nanoaggregates secondary detection reagents
11. Mix well and incubate on ice for 60 min.
12. Centrifuge at 400×g for 5 min and remove suspension and add fresh 1.5 ml of 1×PBS in Eppendorf tubes. Repeat once (wash twice).
13. Now transfer the suspension to round bottom propylene flow tubes. Resuspend the cells in 2 ml of PBS. Add PBS to make the total volume 3 ml.
14. Acquire the data by using BD FACSCalibur flow cytometer with the fluorescence emission at 585 nm by FL2 (Ex 488 nm/Em 585 nm) and 705 nm by FL3 channel (Ex 488 nm/Em 670 nm).

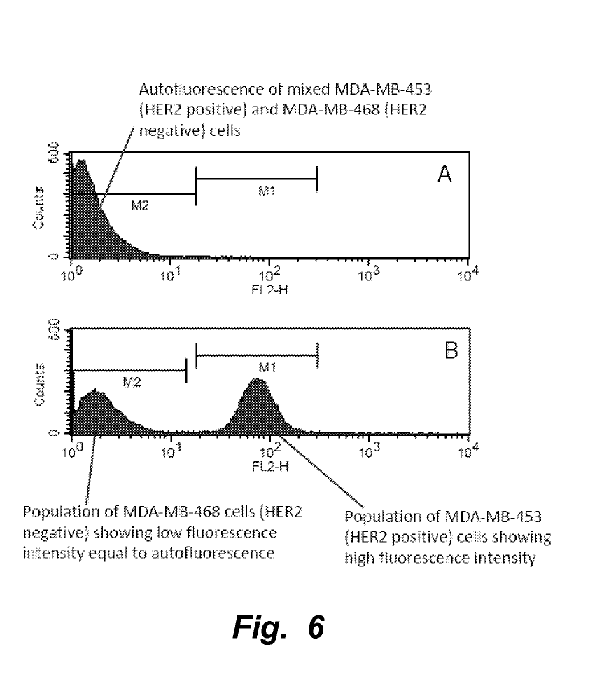

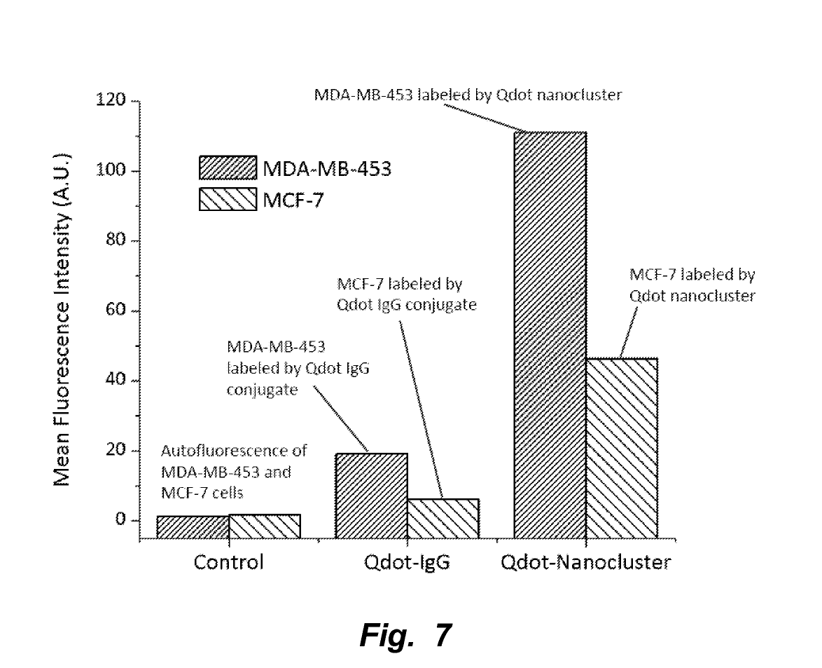

Flow cytometry of live breast tumor cells MDA-MB-453 (high HER2 expression) and MDA-MB-468 (HER2 negative) are shown in FIGS. 6-7. The fluorescence intensities correctly reflect the HER2 receptor expression levels in these two cell lines.

### Example 3

### Method and Protocol for Immunostaining FFPE Sections Using Targeted Nanoclusters or Nanoaggregates

An exemplary protocol using the present composition and method for labeling cells in formalin-fixed, paraffin-embedded sections is described. FFPE slides coated with HER2 human breast carcinoma cells were stained to visualize the erbB2 receptor as follows:

### Part 1.

1. Bake slides in oven at 60° C. for 30 minutes prior to staining
2. Deparaffinize and Rehydrate the tissues on slides
3. Incubate with Ficin for 10 minutes at 37° C.
4. Wash in PBS for 3.5 minutes three times
5. Block in 3% H₂O₂ for 15 minutes
6. Wash in PBS for 3.5 minutes three times
7. Incubate with normal goat serum for 30 minutes at room temperature
8. Incubate with erbB2 antibody (primary antibody)

### Part 2.

9. Wash off coverslips with PBS for 8 minutes
10. Wash in PBS for 3.5 minutes twice
11. Incubate with goat anti-mouse targeted nanoclusters or nanoaggregates for 30 minutes at room temperature
12. Wash in PBS for 3.5 minutes three times
13. Apply coverslip with mounting medium
14. Inspect and readout by automated high-speed scanning instrument

Required processing time is approximately 4-8 hours (partially depending on the requirement of primary antibody binding) and may be performed by a medical/biological laboratory assistant with general bench experience. A single reagent replaces the secondary antibody, colorimetric/fluorescent labeling agent, and even counterstaining

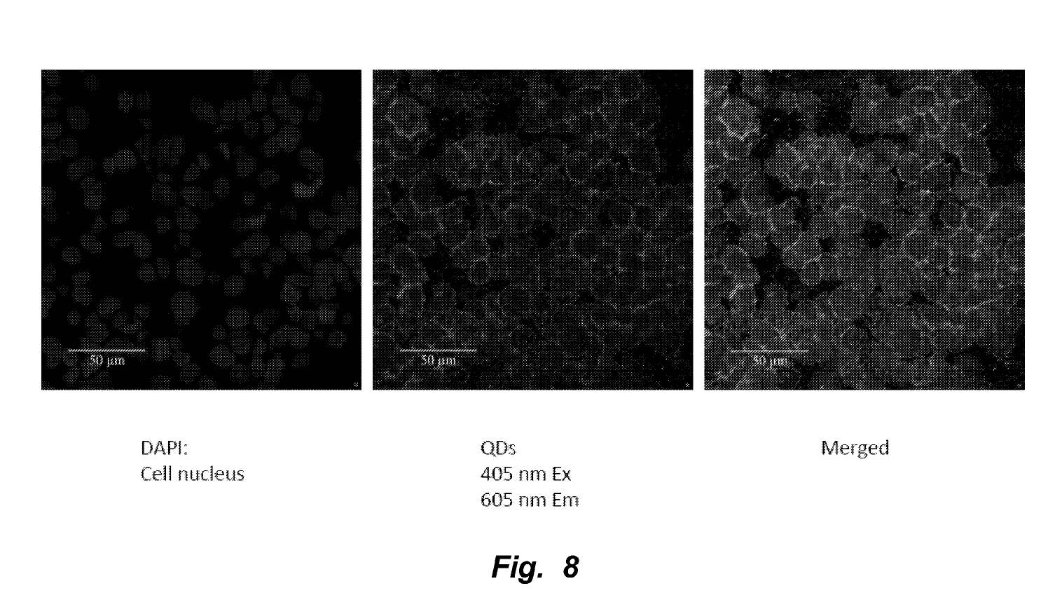

Immunostaining results for fluorescence microscopy images of formalin-fixed, paraffin-embedded (FFPE) sections of human breast cancer cells SK-BR-3, MCF-7, MDA-MB-468, using the targeted nanocluster or nanoaggregate are shown in FIGS. 8-10.

Referring now to FIG. 8, Sk-BR-3 cells were fixed and processed into 5 μm FFPE sections. The sections were then subjected to immunostaining procedure as described above.

The section was then examined by fluorescence microscopy and the image showed erbB2-targeting nanocluster or nanoaggregate binding to the HER2 receptors with strong fluorescence delineating the cell membranes. The results can be evaluated by pathologists or analyzed by available digital pathology software such as those developed by Aperio (on the internet at aperio.com/), Definiens (on the internet at definiens.com/), PDS Pathology Data Systems (on the internet at pds-america.com/), Elekta Impac Software (on the internet at elekta.com/healthcare international impac software.php), Biomedical Photometrics (on the internet at confocal.com/) that are capable of setting proper thresholds for fluorescence intensity with correlation to quantitative erbB2 expression levels. This can also be used as the control reference/standard for erbB2 immunostaining by targeted nanocluster. Patient tumor samples immunostaining can be compared to the cell buttons and extrapolate for quantitative interpretation. Panels from left to right: DAPI staining for cell nucleus; fluorescence emission at 605 nm by 405 nm excitation, indicating distribution of targeted quantum dot nanocluster; and the merged images.

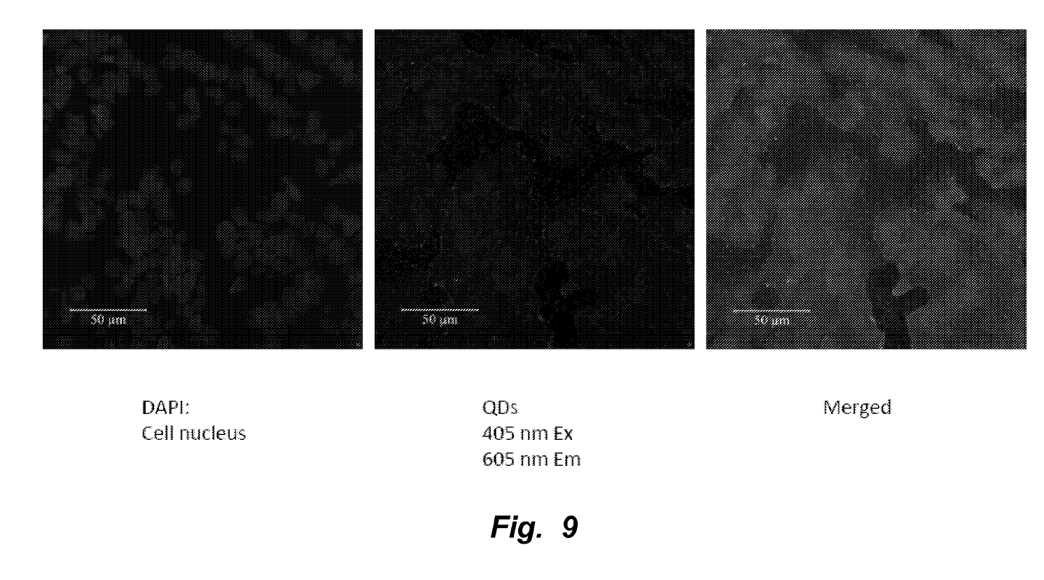

Referring now to FIG. 9, fluorescence microscopy images for MCF-7 cells are shown. MCF-7 represents the low erbB2 expressing cell line with an average erbB2 receptor level ˜10⁴ per cell.^{20, 24} MCF-7 cells were processed and inspected by the same method described above for SK-BR-3 cells. The image showed that the targeted nanocluster or nanoaggregate bound to MCF-7 cell membranes to a much lesser extent, with weaker fluorescence visible from the cell peripherals. Panels from left to right: DAPI staining for cell nucleus; fluorescence emission at 605 nm by 405 nm excitation, indicating distribution of targeted quantum dot nanocluster; and the merged images.

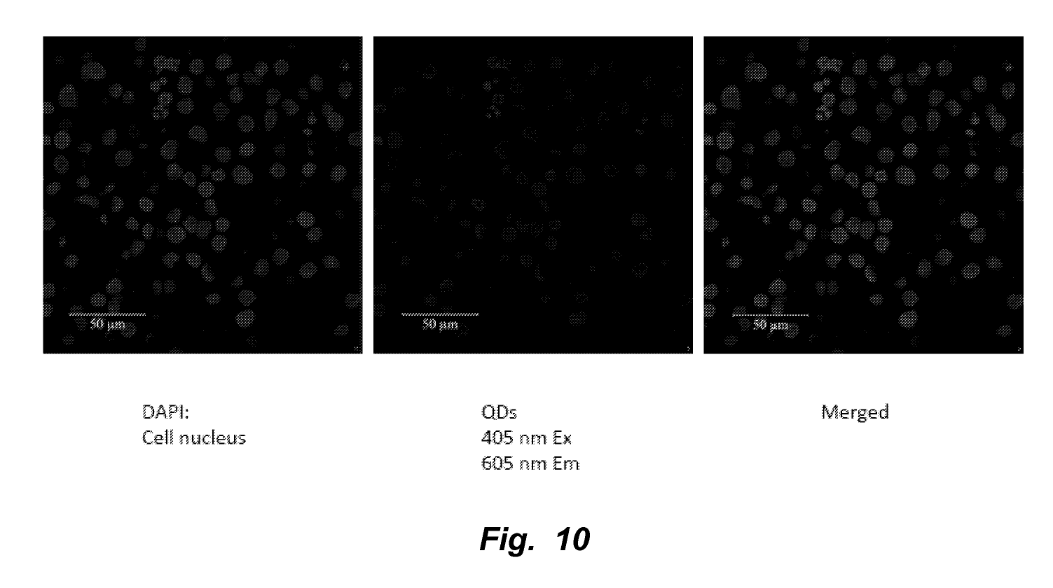

Referring now to FIG. 10, MDA-MB-468 cells were processed and inspected by the same method described above for SK-BR-3 cells. The fluorescence microscopy image showed that amount of targeted nanocluster or nanoaggregate bound to MDA-MB-468 cell membranes was negligible. Together with SK-BR-3 and MCF-7, the results validated targeted nanocluster or nanoaggregate across the range of erbB2 expression from high to negative cell lines, and can be used to establish reference standards for comparison and staining results.

The results show that sensitive and quantitative detection of tumor biomarkers through localization of luminescent quantum dots (QDs) was achieved. Targeted quantum dot nanoclusters or nanoaggregates specifically and efficiently bound to the target antigens. The fluorescence images and intensities reflect the localization of antigens and indicate the expression levels of the antigens, respectively. By attaching a multiplicity of QDs, amplification of signals was realized. Furthermore, by employing a fluorescence imaging modality, the differentiation of targeted nanocluster or nanoaggregate binding across the range of erbB2 expression from high to negative cell lines indicated the dynamic range of detection is improved compared to traditional chromogenic approaches.

### REFERENCES

1. Rimm, Nature Biotechnology 2006, 24, (8), 914-916.
2. Neve, et al., Cancer Cell 2006, 10, (6), 515-527.
3. Kang, et al., Current Opinion in Obstetrics and Gynecology 2008, 20, (1), 40-46.
4. Rakha, et al., Journal of Clinical Oncology 2008, 26, (15), 2568-2581.
5. Dabbs, D. J., Diagnostic Immunohistochemistry. 2nd ed.; Churchill Livingstone: 2006.
6. Jaiswal, et al., Trends in Cell Biology 2004, 14, (9), 497-504.
7. Jaiswal, et al., Nature Methods 2004, 1, (1), 73-78.
8. Bruchez, Current Opinion in Chemical Biology 2005, 9, (5), 533-537.
9. Medintz, et al., Nature Materials 2005, 4, (6), 435-446.
10. Michalet, et al., Science 2005, 307, (5709), 538-544.
11. So, et al., Nature Biotechnology 2006, 24, (3), 339-343.
12. Smith, et al., Nature Biotechnology 2009, 27, 732-733.
13. Wang, et al., Nature 2009, 459, (7247), 686-689.
14. Xing, et al., Nature Protocols 2007, 2, (5), 1152-1165.
15. Bandura, et al., Analytical Chemistry 2009, 81, (16), 6813-6822.
16. Weng, et al., Nano Letters 2008, 8, (9), 2851-2857.
17. Iden, et al., Biochimica et Biophysica Acta (BBA)—Biomembranes 2001, 1513, (2), 207-216.
18. Noble, et al., Expert Opinion on Therapeutic Targets 2004, 8, (4), 335-353.
19. Hong, et al., Analytical Biochemistry 2009, 384, (2), 368-370.
20. Park, et al., Proc. Natl. Acad. Sci. USA 1995, 92, (5), 1327-1331.
21. Kirpotin, et al., Biochemistry 1997, 36, (1), 66-75.
22. Wu, et al., Proc. Natl. Acad. Sci. USA 2009, 106, (27), 10917-10921.
23. O'Neill, et al., Proc. Natl. Acad. Sci. USA 2006, 103, (44), 16153-16158.
24. Park, et al., Journal of Controlled Release 2001, 74, (1-3), 95-113.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A composition comprising a population of nanoclusters, the preponderance of nanoclusters in said population comprising a plurality of crosslinked nanoparticles, said nanoparticles comprising a nanoscaffold core structure having attached thereto:
a targeting moiety; and
a detectable label;
wherein the average number of nanoparticles in a nanocluster in said composition is about 2 or more.

2. (canceled)

3. The composition of claim 1, wherein the median number or average number of nanoparticles in a nanocluster in said composition is about 2, about 3 or more, about 4 or more, about 5 or more, about 6 or more, about 7 or more, about 8 or more, about 9 or more, or about 10 or more.

4. The composition of claim 1, wherein said nanoscaffold core structures bear on average at least 2, or at least 3, or at least 4, or at least 5, or at least 10, or at least 20, or at least 50, or at least 100 or at least 500, or at least 1000 targeting moieties.

5. The composition of claim 4, wherein the targeting moieties are all the same.

6. The composition of claim 4, wherein the targeting moieties comprise a plurality of different targeting moieties.

7. The composition of claim 6, wherein the targeting moieties attached to a nanoscaffold comprise at least two different targeting moieties that bind different targets/epitopes on a target cell.

8. The composition of claim 1, wherein said nanoscaffold core structures bear on average at least 2, or at least 3, or at least 4, or at least 5, or at least 10, or at least 20, or at least 50, or at least 100 or at least 500, or at least 1000 detectable labels.

9. The composition of claim 8, wherein the detectable labels are all the same.

10. The composition of claim 8, wherein the detectable labels comprise a plurality of different detectable labels.

11. The composition of claim 8, wherein the detectable labels attached to a nanoscaffold comprise at least two different detectable labels, each label detectable by a different detection modality.

12. The composition of claim 1, wherein the nanoscaffold core structure is selected from the group consisting of a lipidic particle, a dendrimer, a hyperbranched polymer, a metal particle, a particle comprising a group II, III, or IV material, a polymeric nanoparticle, a glass nanoparticle, a quartz nanoparticle, a viral nanoparticle, a silicon oxide nanoparticle and a silica nanoparticle.

13. The composition of claim 12, wherein the nanoscaffold core structure comprises a lipidic particle selected from the group consisting of a liposome, a micelle, a lipid vesicle and a multilamellar vesicle.

14. The composition of claim 1, wherein said targeting moiety specifically or preferentially binds to a cancer or tumor marker.

15. The composition of claim 14, wherein said targeting moiety selectively or preferentially binds to a cancer marker selected from Her2/neu, 5-alpha reductase, α-fetoprotein, AM-1, APC, APRIL, BAGE, β-catenin, Bc12, bcr-abl (b3a2), CA 125, CASP-8/FLICE, Cathepsins, CD19, CD20, CD21, CD23, CD22, CD38, CD33, CD35, CD44, CD45, CD46, CD5, CD52, CD55, CD59 (791Tgp72), CDC27, CDK4, CEA, c-myc, COX-2, Cytokeratin, DCC, DcR3, E6/E7, EGFR, EMBP, Ena78, Estrogen Receptor (ER), FGF8b and FGF8a, FLK 1/KDR, Folic Acid Receptor, G250, GAGE-Family, gastrin 17, Gastrin-releasing hormone (bombesin), GD2/GD3/GM2, GnRH, GnTV, gp100/Pmel17, gp-100-in4, gp15, gp75/TRP-1, hCG, Heparanase, Her3, HMTV, Hsp70, hTERT (telomerase), IGFR1, IL 13R, iNOS, Ki 67, KIAA0205, K-ras, H-ras, N-ras, KSA (CO17-1A), LDLR-FUT, MAGE Family (MAGE1, MAGE3, etc.), Mammaglobin, MAP17, Melan-A/MART-1, mesothelin, MIC A/B, MT-MMP's, such as MMP2, MMP3, MMP7, MMP9, Mox1, Mucin, such as MUC-1, MUC-2, MUC-3, and MUC-4, MUM-1, NY-ESO-1, Osteonectin, p15, P170/MDR1, p53, p97/melanotransferrin, PAI-1, PDGF, Plasminogen (uPA), PRAME, Probasin, Progenipoietin, Progesterone Receptor (PR), PSA, PSM, RAGE-1, Rb, RCAS1, SART-1, SSX gene family, STAT3, STn (mucin assoc.), TAG-72, TGF-α, TGF-β, Thymosin β-15, IFN-γ, TPA, TPI, TRP-2, Tyrosinase, VEGF, ZAG, p16INK4, Glutathione and S-transferase.

16. The composition of claim 1, wherein said targeting moiety specifically or preferentially binds to a cell from a cancer selected from the group consisting of breast cancer, colorectal cancer, NSCLC, lung cancer, bone cancer, pancreatic cancer, skin cancer, cancer of the head or neck, cutaneous melanoma, intraocular melanoma, uterine cancer, ovarian cancer, rectal cancer, anal region cancer, stomach cancer, gastric cancer, colon cancer, breast cancer, uterine cancer, fallopian tube carcinoma, endometrial carcinoma, cervical carcinoma, vaginal carcinoma, vulval carcinoma, Hodgkin's Disease, esophagus cancer, small intestine cancer, endocrine system cancer, thyroid gland cancer, parathyroid gland cancer, adrenal gland cancer, soft tissue sarcoma, urethral cancer, penis cancer, prostate cancer, bladder cancer, kidney cancer, ureter cancer, renal cell carcinoma, renal pelvis carcinoma, mesothelioma, hepatocellular cancer, biliary cancer, chronic leukemia, acute leukemia, lymphocytic lymphoma, CNS neoplasm, spinal axis cancer, brain stem glioma, glioblastoma multiform, astrocytoma, schwannoma, ependymoma, medulloblastoma, meningioma, squamous cell carcinoma and pituitary adenoma tumors, and tumor metastasis.

17. The composition of claim 1, wherein said targeting moiety specifically or preferentially binds to Her2/neu, and said cell is a cell from a breast cancer; or wherein said targeting moiety specifically or preferentially binds to a primary antibody, and said primary antibody specifically binds to HER2/neu is a cell from a breast cancer.

18. (canceled)

19. The composition of claim 1, wherein said targeting moiety specifically or preferentially binds to a stem cell or blood cell marker.

20. The composition of claim 1, wherein said targeting moiety specifically or preferentially binds to a stem cell biomarker selected from the group consisting of ABCG2, alpha 6, beta 1, B-catenin, C-myc, CK14, CK15, Ck19, CD34, CD71, CD117, CD133, Nestin, Oct-4, p63, p75 Neurotrophin R, NCAM, Sca-1, STRO-1.

21. The composition of claim 1, wherein said targeting moiety specifically or preferentially binds to the Fc portion of an immunoglobulin.

22. The composition of claim 1, wherein said targeting moiety is selected from the group consisting of an antibody or antibody fragment, a unibody, an affybody, an aptamer, a ligand, and a polynucleotide.

23. The composition of claim 22, wherein said antibody is an antibody selected from the group consisting of an IgG, an scFv, an Fv, an Fab, an Fab′, an F(ab′)₂, a bis-scFv, heavy-light chains, a monoclonal antibody, a polyclonal antibody, a single domain antibody, a nanobody, a minibody, a diabody, a triabody, or a tetrabody.

24. The composition of claim 1, wherein the detectable label is selected from the group consisting of a fluorescent label, an enzyme, a colorimetric label, a luminescent label, a radioactive label, a contrast agent, an MRI label, an electron spin label, and a magnetic label.

25. The composition of claim 1, wherein the detectable label comprises a fluorescent nanostructure or a radioactive label.

26. The composition of claim 25, wherein the detectable label comprises a fluorescent nanostructure selected from the group consisting of a quantum dot, a quantum rod and a quantum wire.

27. (canceled)

28. The composition of claim 25, wherein the detectable label comprises a radioactive label selected from the group consisting of ³H, ¹²⁵I, ³⁵S, ¹⁴C, ³²P, ⁹⁹Tc, ²⁰³Pb, ⁶⁷Ga, ⁶⁸Ga, ⁷²As, ¹¹¹In, ^{113m}In, ⁹⁷Ru, ⁶²Cu, ⁶⁴Cu, ⁵²Fe, ^{52m}Mn, ⁵¹Cr, ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, and ¹¹¹Ag.

29. (canceled)

30. The composition of claim 1, wherein:
said targeting moiety comprises an antibody;
said nanoscaffold core structure comprises a liposome; and
said detectable label comprises a quantum dot.

31. The composition of claim 30, wherein said antibody specifically binds Her2/neu.

32. The composition of claim 30, wherein said antibody specifically binds to the Fc portion of an antibody.

33. A method of detecting the presence of and/or quantifying a biomarker, said method comprising:
a) contacting a subject or a biological sample suspected of containing the biomarker with a population of nanoclusters of claim 1; and
b) detecting the detectable label of the bound nanoclusters, whereby the presence of bound nanoclusters indicates the presence of and/or quantifies the biomarker.

34. The method of claim 33, wherein said contacting a subject or a biological sample comprises administering the population of nanoclusters to the subject.

35-37. (canceled)

38. The method of claim 33, wherein said detecting comprises using a detection modality selected from the group consisting of x-ray imaging, CAT scanning, MRI, PET, electron spin resonance (ESR) detection, and thermographic imaging.

39. The method of claim 33, wherein said contacting a subject or a biological sample comprises contacting the population of nanoclusters to a biological sample.

40. The method of claim 39, wherein the biological sample comprises a sample selected from the group consisting of blood or a blood fraction, cerebrospinal fluid, urine, saliva, mucus, and a tissue sample.

41. (canceled)

42. The method of claim 39, wherein the population of nanoclusters comprises a detection reagent formulated for use in an application selected from the group consisting of immunohistochemistry, immunocytochemistry, immunohistology, flow cytometry, ELISA, Western blot, dot blot, fluorescent in situ hybridization (FISH), high-resolution capillary isoelectric focusing, secondary ion mass spectrometry, mass cytometry, micro bead assays and solid phase particle-based assays.

43. The method of claim 33, wherein said detecting the presence of and/or quantifying a biomarker comprises detecting or quantifying a tumor or cancer cell.

44. The method of claim 43, wherein said detecting the presence of and/or quantifying a biomarker comprises detecting and/or quantifying a cancer marker selected from selected from Her2/neu, 5-alpha reductase, α-fetoprotein, AM-1, APC, APRIL, BAGE, β-catenin, Bc12, bcr-abl (b3a2), CA 125, CASP-8/FLICE, Cathepsins, CD19, CD20, CD21, CD23, CD22, CD38, CD33, CD35, CD44, CD45, CD46, CD5, CD52, CD55, CD59 (791Tgp72), CDC27, CDK4, CEA, c-myc, COX-2, Cytokeratin, DCC, DcR3, E6/E7, EGFR, EMBP, Ena78, Estrogen Receptor (ER), FGF8b and FGF8a, FLK 1/KDR, Folic Acid Receptor, G250, GAGE-Family, gastrin 17, Gastrin-releasing hormone (bombesin), GD2/GD3/GM2, GnRH, GnTV, gp100/Pmel17, gp-100-in4, gp15, gp75/TRP-1, hCG, Heparanase, Her3, HMTV, Hsp70, hTERT (telomerase), IGFR1, IL 13R, iNOS, Ki 67, KIAA0205, K-ras, H-ras, N-ras, KSA (CO17-1A), LDLR-FUT, MAGE Family (MAGE1, MAGE3, etc.), Mammaglobin, MAP17, Melan-A/MART-1, mesothelin, MIC A/B, MT-MMP's, such as MMP2, MMP3, MMP7, MMP9, Mox1, Mucin, such as MUC-1, MUC-2, MUC-3, and MUC-4, MUM-1, NY-ESO-1, Osteonectin, p15, P170/MDR1, p53, p97/melanotransferrin, PAI-1, PDGF, Plasminogen (uPA), PRAME, Probasin, Progenipoietin, Progesterone Receptor (PR), PSA, PSM, RAGE-1, Rb, RCAS1, SART-1, SSX gene family, STAT3, STn (mucin assoc.), TAG-72, TGF-α, TGF-β, Thymosin β-15, IFN-γ, TPA, TPI, TRP-2, Tyrosinase, VEGF, ZAG, p16INK4, Glutathione and S-transferase.

45. The method of claim 43, wherein said detecting and/or quantifying comprises detecting and/or quantifying a cell from a cancer selected from the group consisting of breast cancer, colorectal cancer, NSCLC, lung cancer, bone cancer, pancreatic cancer, skin cancer, cancer of the head or neck, cutaneous melanoma, intraocular melanoma, uterine cancer, ovarian cancer, rectal cancer, anal region cancer, stomach cancer, gastric cancer, colon cancer, breast cancer, uterine cancer, fallopian tube carcinoma, endometrial carcinoma, cervical carcinoma, vaginal carcinoma, vulval carcinoma, Hodgkin's Disease, esophagus cancer, small intestine cancer, endocrine system cancer, thyroid gland cancer, parathyroid gland cancer, adrenal gland cancer, soft tissue sarcoma, urethral cancer, penis cancer, prostate cancer, bladder cancer, kidney cancer, ureter cancer, renal cell carcinoma, renal pelvis carcinoma, mesothelioma, hepatocellular cancer, biliary cancer, chronic leukemia, acute leukemia, lymphocytic lymphoma, CNS neoplasm, spinal axis cancer, brain stem glioma, glioblastoma multiform, astrocytoma, schwannoma, ependymoma, medulloblastoma, meningioma, squamous cell carcinoma and pituitary adenoma tumors, and tumor metastasis.

46. The method of claim 33, wherein said detecting the presence of and/or quantifying a biomarker comprises detecting or quantifying a stem cell or a blood cell.

47. A method of producing a population of nanoclusters of claim 1, said method comprising:
a) providing a nanoscaffold with at least a first functional group and a second functional group, wherein the first and second functional groups are different from each other and are suitable for crosslinking or conjugation;
b) attaching a targeting moiety to the first functional group;
c) attaching a detectable moiety to the second functional group; wherein steps b) and c) can be performed in either order; and
d) crosslinking between two or more nanoscaffolds.

48. The method of claim 47, wherein crosslinking between two or more nanoscaffolds occurs concurrently with either step b) or step c), thereby producing a population of nanoclusters.

49. The method of claim 47, wherein crosslinking between two or more nanoscaffolds is performed separately from steps b) and c).

50. The method of claim 47, wherein the targeting moiety is crosslinked or conjugated to the first functional group.

51. The method of claim 47, wherein the detectable moiety is crosslinked or conjugated to the second functional group.
